SEQUENCE LISTING (3 sequences)

SEQ ID NO: 1 - protein, 2505 aa, Homo sapiens
MARFGDEMPARYGGGGSGAAAGVVVGSGGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAAWGRMPYLDMYQMLRHMSPPLGLGKKCPARVAYKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGPPQQQQQQQQQQQAVARPGRAATSGPRRYPGPTAEPLAGDRPPTGGHSSGRSPRMERRVPGPARSESPRACRHGGARWPASGPHVSEGPPGPRHHGYYRGSDYDEADGPGSGGGEEAMAGAYDAPPPVRHASSGATGRSPRTPRASGPACASPSRHGRRLPNGYYPAHGLARPRGPGSRKGLHEPYSESDDDWC

SEQ ID NO: 2 - protein, 1145 aa, Homo sapiens
  MISC_FEATURE (1)..(1145): PQ calcium channel, alpha 2/delta subunit 2 isoform a
MAVPARTCGASRPGPARTARPWPGCGPHPGPGTRRPTSGPPRPLWLLLPLLPLLAAPGASAYSFPQQHTMQHWARRLEQEVDGVMRIFGGVQQLREIYKDNRNLFEVQENEPQKLVEKVAGDIESLLDRKVQALKRLADAAENFQKAHRWQDNIKEEDIVYYDAKADAELDDPESEDVERGSKASTLRLDFIEDPNFKNKVNYSYAAVQIPTDIYKGSTVILNELNWTEALENVFMENRRQDPTLLWQVFGSATGVTRYYPATPWRAPKKIDLYDVRRRPWYIQGASSPKDMVIIVDVSGSVSGLTLKLMKTSVCEMLDTLSDDDYVNVASFNEKAQPVSCFTHLVQANVRNKKVFKEAVQGMVAKGTTGYKAGFEYAFDQLQNSNITRANCNKMIMMFTDGGEDRVQDVFEKYNWPNRTVRVFTFSVGQHNYDVTPLQWMACANKGYYFEIPSIGAIRINTQEYLDVLGRPMVLAGKEAKQVQWTNVYEDALGLGLVVTGTLPVFNLTQDGPGEKKNQLILGVMGIDVALNDIKRLTPNYTLGANGYVFAIDLNGYVLLHPNLKPQTTNFREPVTLDFLDAELEDENKEEIRRSMIDGNKGHKQIRTLVKSLDERYIDEVTRNYTWVPIRSTNYSLGLVLPPYSTFYLQANLSDQILQVKYFEFLLPSSFESEGHVFIAPREYCKDLNASDNNTEFLKNFIELMEKVTPDSKQCNNFLLHNLILDTGITQQLVERVWRDQDLNTYSLLAVFAATDGGITRVFPNKAAEDWTENPEPFNASFYRRSLDNHGYVFKPPHQDALLRPLELENDTVGILVSTAVELSLGRRTLRPAVVGVKLDLEAWAEKFKVLASNRTHQDQPQKCGPNSHCEMDCEVNNEDLLCVLIDDGGFLVLSNQNHQWDQVGRFFSEVDANLMLALYNNSFYTRKESYDYQAACAPQPPGNLGAAPRGVFVPTVADFLNLAWWTSAAAWSLFQQLLYGLIYHSWFQADPAEAEGSPETRESSCVMKQTQYYFGSVNASYNAIIDCGNCSRLFHAQRLTNTNLLFVVAEKPLCSQCEAGRLLQKETHCPADGPEQCELVQRPRYRRGPHICFDYNATEDTSDCGRGASFPPSLGVLVSLQLLLLLGLPPRPQPQVLVHASRRL

SEQ ID NO: 3 - protein, 598 aa, Homo sapiens
  MISC_FEATURE (1)..(598): channel beta subunit PQ
MVQKTSMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQKLRQNRLGSSKSGDNSSSSLGDVVTGTRRPTPPASAKQKQKSTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSSTPPNPLLNRTMATAALAASPAPVSNLQGPYLASGDQPLERATGEHASMHEYPGELGQPPGLYPSSHPPGRAGTLRALSRQDTFDADTPGSRNSAYTELGDSCVDMETDPSEGPGLGDPAGGGTPPARQGSWEDEEEDYEEELTDNRNRGRNKARYCAEGGGPVLGRNKNELEGWGRGVYIR

(19) United States
(12) Patent Application Publication
(10) Pub. No.: US 2015/0079096 A1
(43) Pub. Date: Mar. 19, 2015

(54) METHOD FOR THE TREATMENT OF AMYLOIDOSES

(71) Applicant: AbbVie Deutschland GmbH & Co. KG, Wiesbaden (DE)

(72) Inventors: Volker Nimmrich, Heidelberg (DE); Stefan Barghorn, Mannheim (DE); Ulrich Ebert, Mannheim (DE); Heinz Hillen, Hassloch (DE); Gerhard Gross, Speyer (DE); Andreas Draguhn, Heidelberg (DE); Claus Bruhl, Schonau (DE); Christiane Grimm, Leimen (DE); Carsten Krantz, Allschwil (DE)

(21) Appl. No.: 14/514,168

(22) Filed: Oct. 14, 2014

Related U.S. Application Data
(62) Division of application No. 12/529,467, filed on Jul. 27, 2010, now Pat. No. 8,895,004.
(60) Provisional application No. 60/903,695, filed on Feb. 27, 2007.

(30) Foreign Application Priority Data
Oct. 16, 2007 (EP) 07020258.5
Jan. 9, 2008 (EP) 08000324.7

(51) Int. Cl. C07K 16/28; G01N 33/68

(52) U.S. Cl. CPC C07K 16/28; G01N 33/6896; C07K 2317/76; A61K 2039/505
USPC 424/139.1; 435/7.21

(57) ABSTRACT

The present invention relates to a method for the treatment of an amyloidosis such as Alzheimer's disease in a subject in need thereof, characterized in that it comprises administering an inhibitor of the interaction between Aβ globulomer and the P/Q type voltage-gated presynaptic calcium channel to said subject.

The present invention relates to a method for the treatment of an amyloidosis such as Alzheimer's disease.

Alzheimer's disease (AD), the most frequent cause for dementia among the aged with an incidence of about 10% of the population above 65 years, is a dementing disorder characterized by a progressive loss of cognitive abilities and by characteristic neuro-pathological features comprising extracellular amyloid deposits, intracellular neuro-fibrillary tangles and neuronal loss in several brain regions (Mattson, M. P. Pathways towards and away from Alzheimer's disease. Nature 430, 631-639 (2004); Hardy, J. & Selkoe, D. J. The amyloid hypothesis of Alzheimer's disease: progress and problems on the road to therapeutics. Science 297, 353-356 (2002)). The principal constituents of the amyloid deposits are amyloid β peptides (Aβ) which arise from the β-amyloid precursor protein (APP) by proteolytic cleavage.

Both cerebral amyloid deposits and cognitive impairments very similar to those observed in Alzheimer's disease are also hallmarks of Down's syndrome (trisomy 21), which occurs at a frequency of about 1 in 800 births. Hence, Alzheimer's disease and Down's syndrome are jointly termed “amyloidoses”.

Recently, however, it was shown that in amyloidoses soluble, globular Aβ oligomers, rather than the eponymous insoluble amyloid deposits, are the causative agents for the impairment of higher-level functions, such as memory function, as indicated by its sup-pressing effect on long-term potentiation (WO2004/067561; Barghom S. et al., J. Neurochem. 95: 834-847 (2005); WO2006/094724).

The term “Aβ globulomer” here refers to a particular soluble, globular, non-covalent association of Aβ peptides, possessing homogeneity and distinct physical characteristics. Aβ globulomers are stable, non-fibrillar, oligomeric assemblies of Aβ peptides which are obtainable by incubation with anionic detergents, in particular as described in WO2004/067561. In contrast to Aβ monomer and fibrils, these globulomers are characterized by defined assembly numbers of subunits (WO2004/067561). The globulomers have a characteristic three-dimensional globular type structure (“molten globule”, see Barghorn et al., J. Neurochem. 95: 834-847 (2005)). They have been shown to closely mimic the properties, behaviour and effects of naturally occurring soluble Aβ oligomers.

Soluble Aβ oligomer was found to impair the functioning of the central nervous system even before the onset of cytotoxicity. However, the exact mechanisms whereby soluble Aβ oligomer causes memory failure in amyloidoses has not been elucidated so far, and a lack of understanding of such mechanisms has so far hampered the development of rational therapeutic approaches for inhibiting the further progression of the disease or compensating the damage already done.

It was thus an object of the present invention to provide a new approach to the treatment of amyloidoses such as Alzheimer's disease, in particular to rehabilitating treatment such as the restoration of cognitive abilities in amyloidoses such as Alzheimer's disease.

Surprisingly, it was now found that Aβ globulomer exerts its detrimental effects essentially by hampering normal ion fluxes through the P/Q type presynaptic calcium channel, reducing presynaptic neurotransmitter release and inhibiting spontaneous synaptic activity and thereby interfering with the proper functioning of the central nervous sys-tern even before the onset of manifest neural cytotoxicity, and that inhibition of the interaction of the Aβ globulomer with the P/Q type presynaptic calcium channel is therefore effective in compensating these effects.

In a first aspect the present invention thus relates to a method for the treatment of an amyloidosis, preferably Alzheimer's disease, in a subject in need thereof, comprising administering an inhibitor of the interaction between Aβ globulomer and the P/Q type voltage-gated presynaptic calcium channel (hereinafter referred to as “Aβ-P/Q interaction”) to said subject.

The P/Q type voltage-gated presynaptic calcium channel (the channel is also referred to as Caᵥ2.1 channel and the associated currents as P/Q type currents) belongs to the group of voltage-gated calcium channels which mediate the influx of calcium ions into excitable cells. The opening state of a voltage-gated channel is controlled by the electrical state of the surrounding membrane; however, the responsiveness of the P/Q type voltage-gated presynaptic calcium channel to membrane depolarization is extensively modulated, both qualitatively and quantitatively, by and/or through its interaction partners.

As used herein, a “P/Q type voltage-gated presynaptic calcium channel” is a voltage-gated calcium channel that is functionally characterized by its sensitivity towards ω-agatoxin IVA (a well-known funnel web spider venom).

According to a particular embodiment, ω-agatoxin IVA acts as a gating modifier of the P/Q type voltage-gated presynaptic calcium channel (e.g., P type Kd=1-3 nM; Q type Kd=100-200 nM). Further, P/Q type voltage-gated presynaptic calcium channels according to the present invention may be characterized by one or more than one of the following features:
(i) requires strong depolarization for activation (high-voltage activation); and
(ii) no or slow inactivation.

The P/Q type voltage-gated presynaptic calcium channel according to the present invention comprises an α1 subunit. According to a particular embodiment of the invention, the α1 subunit has an amino acid sequence with at least 70%, advantageously at least 80%, preferably at least 90%, more preferably at least 95% and in particular at least 98%, e. g. at least 99%, amino acid sequence identity with the sequence SEQ ID NO: 1. The α1 subunit incorporates the conduction pore, the voltage sensor and gating apparatus, and sites of channel regulation by second messengers, drugs, and toxins.

Usually, the P/Q type voltage-gated presynaptic calcium channel also comprises an α2-δ subunit and a β subunit. It may also comprise any subunit. In a particular embodiment of the invention, the α2-δ subunit, when present, has at least 70%, advantageously at least 80%, preferably at least 90%, more preferably at least 95% and in particular at least 98%, e. g. at least 99%, amino acid sequence identity with the sequence SEQ ID NO:2. In a further particular embodiment of the invention, the β sub-unit, when present, has at least 70%, advantageously at least 80%, preferably at least 90%, more preferably at least 95% and in particular at least 98%, e. g. at least 99%, amino acid sequence identity with the sequence SEQ ID NO:3.

Further characteristic features of P/Q type voltage-gated presynaptic calcium channels are described in Catterall W A, Perez-Reyes E, Snutch T P, Striessnig J. International Union of Pharmacology. XLVIII. Nomenclature and structure-function relationships of voltage-gated calcium channels. Pharmacol Rev. 57: 411-25 (2005), which is herein incorporated by reference in its entirety.

The term “Aβ globulomer” here refers to any Aβ(X—Y) globulomer which is a soluble, globular, non-covalent association of Aβ(X—Y) peptides, wherein an Aβ(X—Y) peptide is a fragment of the amyloid β protein from amino acid residue X to amino acid residue Y inclusive, possessing homogeneity and distinct physical characteristics. According to one aspect, Aβ(X—Y) globulomers are stable, non-fibrillar, oligomeric assemblies of Aβ(X—Y) peptides which are obtainable by incubation with anionic detergents. In contrast to monomer and fibrils, these globulomers are characterized by defined assembly numbers of subunits (e.g. early assembly forms, n=4-6, “oligomers A”, and late assembly forms, n=12-14, “oligomers B”, as described in WO2004/067561). The globulomers have a 3-dimensional globular type structure (“molten globule”, see Barghom et al., 2005, J Neurochem, 95, 834-847). They may be further characterized by one or more of the following features:
cleavability of N-terminal amino acids X-23 with promiscuous proteases (such as thermolysin or endoproteinase GIuC) yielding truncated forms of globulomers;
non-accessibility of C-terminal amino acids 24-Y with promiscuous proteases and antibodies;
truncated forms of these globulomers maintain the 3-dimensional core structure of said globulomers with a better accessibility of the core epitope Aβ(X—Y) in its globulomer conformation.

According to the invention and in particular for the purpose of assessing the binding affinities of the antibodies of the present invention, the term “Aβ(X—Y) globulomer” here refers in particular to a product which is obtainable by a process as described in WO 2004/067561, which is incorporated herein by reference.

Said process comprises unfolding a natural, recombinant or synthetic Aβ(X—Y) peptide or a derivative thereof; exposing the at least partially unfolded Aβ(X—Y) peptide or derivative thereof to a detergent, reducing the detergent action and continuing incubation.

For the purpose of unfolding the peptide, hydrogen bond-breaking agents such as, for example, hexafluoroisopropanol (HFIP) may be allowed to act on the protein. Times of action of a few minutes, for example about 10 to 60 minutes, are sufficient when the temperature of action is from about 20 to 50° C. and in particular about 35 to 40° C. Subsequent dissolution of the residue evaporated to dryness, preferably in concentrated form, in suitable organic solvents miscible with aqueous buffers, such as, for example, dimethyl sulfoxide (DMSO), results in a suspension of the at least partially unfolded peptide or derivative thereof, which can be used subsequently. If required, the stock suspension may be stored at low temperature, for example at about −20° C., for an interim period.

Alternatively, the peptide or the derivative thereof may be taken up in slightly acidic, preferably aqueous, solution, for example an about 10 mM aqueous HCl solution. After an incubation time of usually a few minutes, insoluble components are removed by centrifugation. A few minutes at 10000 g is expedient. These method steps are preferably carried out at room temperature, i.e. a temperature in the range from 20 to 30° C. The supernatant obtained after centrifugation contains the Aβ(X—Y) peptide or the de-rivative thereof and may be stored at low temperature, for example at about −20° C., for an interim period.

The following exposure to a detergent relates to the oligomerization of the peptide or the derivative thereof to give an intermediate type of oligomers (in WO 2004/067561 referred to as oligomers A). For this purpose, a detergent is allowed to act on the at least partially unfolded peptide or derivative thereof until sufficient intermediate oligomer has been produced.

Preference is given to using ionic detergents, in particular anionic detergents.

According to a particular embodiment, a detergent of the formula (I):

R—X,

is used, in which

the radical R is unbranched or branched alkyl having from 6 to 20 and preferably 10 to 14 carbon atoms or unbranched or branched alkenyl having from 6 to 20 and preferably 10 to 14 carbon atoms,

the radical X is an acidic group or salt thereof, with X being preferably selected from among —COO⁻M⁺, —SO₃⁻M⁺, and especially

—OSO₃⁻M⁺ and M⁺ is a hydrogen cation or an inorganic or organic cation preferably selected from alkali metal and alkaline earth metal cations and ammonium cations.

Advantageous are detergents of the formula (I)₁ in which R is unbranched alkyl of which alk-1-yl radicals must be mentioned in particular. Particular preference is given to sodium dodecyl sulfate (SDS). Laurie acid and oleic acid can also be used advantageously. The sodium salt of the detergent lauroylsarcosin (also known as sarkosyl NL-30 or Gardol®) is also particularly advantageous.

The time of detergent action in particular depends on whether—and if yes, to what extent—the peptide or the derivative thereof subjected to oligomerization has unfolded. If, according to the unfolding step, the peptide or derivative thereof has been treated beforehand with a hydrogen bond-breaking agent, i.e. in particular with hexafluoroisopro-panol, times of action in the range of a few hours, advantageously from about 1 to 20 and in particular from about 2 to 10 hours, are sufficient when the temperature of action is about 20 to 50° C. and in particular about 35 to 40° C. If a less unfolded or an essentially not unfolded peptide or derivative thereof is the starting point, correspondingly longer times of action are expedient.

If the peptide or the derivative thereof has been pretreated, for example, according to the procedure indicated above as an alternative to the HFIP treatment or said peptide or derivative thereof is directly subjected to oligomerization, times of action in the range from about 5 to 30 hours and in particular from about 10 to 20 hours are sufficient when the temperature of action is about 20 to 50° C. and in particular about 35 to 40° C. After incubation, insoluble components are advantageously removed by centrifugation. A few minutes at 10000 g is expedient.

The detergent concentration to be chosen depends on the detergent used. If SDS is used, a concentration in the range from 0.01 to 1% by weight, preferably from 0.05 to 0.5% by weight, for example of about 0.2% by weight, proves expedient. If lauric acid or oleic acid are used, somewhat higher concentrations are expedient, for example in a range from 0.05 to 2% by weight, preferably from 0.1 to 0.5% by weight, for example of about 0.5% by weight.

The detergent action should take place at a salt concentration approximately in the physiological range. Thus, in particular NaCl concentrations in the range from 50 to 500 mM, preferably from 100 to 200 mM and particularly at about 140 mM are expedient.

The subsequent reduction of the detergent action and continuation of incubation relates to a further oligomerization to give the Aβ(X—Y) globulomer of the invention (in WO 2004/067561 referred to as oligomers B). Since the composition obtained from the preceding step regularly contains detergent and a salt concentration in the physiological range it is then expedient to reduce detergent action and, preferably, also the salt concentration. This may be carried out by reducing the concentration of detergent and salt, for example, by diluting, expediently with water or a buffer of lower salt concentration, for example Tris-HCl, pH 7.3. Dilution factors in the range from about 2 to 10, advantageously in the range from about 3 to 8 and in particular of about 4, have proved suitable. The reduction in detergent action may also be achieved by adding substances which can neutralize said detergent action. Examples of these include substances capable of complexing the detergents, like substances capable of stabilizing cells in the course of purification and extraction measures, for example particular EO/PO block copolymers, in particular the block copolymer under the trade name Pluronic® F 68. Alkoxylated and, in particular, ethoxylated alkyl phenols such as the ethoxylated t-octylphenols of the Triton® X series, in particular Triton® X100, 3-(3-cholamidopropyl-dimethylammonio)-1-propanesulfonate (CHAPS®) or alkoxylated and, in particular, ethoxylated sorbitan fatty esters such as those of the Tween® series, in particular Tween® 20, in concentration ranges around or above the particular critical micelle concentration, may be equally used.

Subsequently, the solution is incubated until sufficient Aβ(X—Y) globulomer of the invention has been produced. Times of action in the range of several hours, preferably in the range from about 10 to 30 hours and in particular in the range from about 15 to 25 hours, are sufficient when the temperature of action is about 20 to 50° C. and in particu-lar about 35 to 40° C. The solution may then be concentrated and possible residues may be removed by centrifugation. Here too, a few minutes at 10000 g proves expedient. The supernatant obtained after centrifugation contains an Aβ(X—Y) globulomer of the invention.

An Aβ(X—Y) globulomer of the invention can be finally recovered in a manner known per se, e. g. by ultrafiltration, dialysis, precipitation or centrifugation.

It is further preferred if electrophoretic separation of the Aβ(X—Y) globulomers under denaturing conditions, e. g. by SDS-PAGE, produces a double band (e. g. with an ap-parent molecular weight of 38/48 kDa for Aβ(1-42)), and especially preferred if upon glutardialdehyde treatment of the globulomers before separation these two bands are merged into one. It is also preferred if size exclusion chromatography of the globulomers results in a single peak (e. g. corresponding to a molecular weight of approximately 100 kDa for Aβ(1-42) globulomer or of approximately 60 kDa for glutardialde-hyde cross-linked Aβ(1-42) globulomer), respectively.

For the purposes of the present invention, an Aβ globulomer is in particular the Aβ(1-42) globulomer as described in reference example 2 herein.

As used herein, an “inhibitor of Aβ-P/Q interaction” is any substance that effectively reduces an Aβ-P/Q interaction and thereby the inhibition of the activity of the P/Q type voltage-gated presynaptic calcium channel by an Aβ globulomer. Preferably, the inhibitor of the Aβ-P/Q interaction exerts no significant effect on activity of the P/Q type voltage-gated presynaptic calcium channel in the absence of Aβ globulomer.

The expression “effectively reduces” is used herein to denote any reduction causally connected with the presence of said inhibitor, irrespective of the individual mode of action.

In a particular embodiment of the invention, an inhibitor of the Aβ-P/Q interaction is a substance that effectively reduces the mutual affinity of Aβ globulomer and the P/Q type voltage-gated presynaptic calcium channel below its normal value, wherein the “normal value” is understood to be the value of [Aβ globulomer-P/Q complex]/([Aβ globulomer]+[P/Q]) in the absence of the inhibitor but under otherwise identical circumstances, which may refer to either molecule being in situ or isolated.

Herein, the term “in situ” is understood to refer to any molecule or structure being in its natural molecular environment as found in an intact cell and/or organism, which may be either healthy or diseased, e. g. as obtainable by taking samples ex vivo, and “isolated” to refer to any molecule or structure essentially separated from at least one of, preferably essentially all of the elements forming its natural environment as found in an intact cell and/or organism, e. g. as obtainable by recombinant expression. Preferably, “isolated” is in vitro.

It is noted that in vivo the P/Q type voltage-gated presynaptic calcium channel may interact with, i.e. bind to, Aβ forms other than the Aβ globulomers described herein. These Aβ forms may or may not be oligomeric or globulomeric. Thus, the ligands with which the P/Q type voltage-gated presynaptic calcium channel interacts include any Aβ form that comprises the globulomer epitope with which Aβ globulomers described herein bind to the P/Q type voltage-gated presynaptic calcium channel. Such Aβ forms include truncated and non-truncated Aβ(X—Y) forms (with X and Y being defined as above), such as Aβ(20-42), Aβ(20-40), Aβ(12-42), Aβ(12-40), Aβ(1-42), and Aβ(1-40) forms, provided that said forms comprise the globulomer epitope.

Inhibitors of the Aβ-P/Q interaction may be identified among compounds known per se by screening for their capacity to prevent and/or reverse the blockade of the P/Q type voltage-gated presynaptic calcium channel caused by Aβ globulomer, preferably by screening using a method comprising determining the effect of a candidate compound on the opening state of the P/Q type voltage-gated presynaptic calcium channel in the presence of Aβ globulomer, most conveniently by determining the effect of said compound on the Ca⁺⁺ flux through the P/Q type voltage-gated presynaptic calcium chan-nel in the presence of Aβ globulomer. Suitable methods for determining transmembrane ion fluxes such as Ca⁺⁺ fluxes through the P/Q type voltage-gated presynaptic calcium channel have been described in the art (Sakmann B and Neher E. Single-Channel Recording. Springer U S, 97 A.D.).

A method for determining whether any candidate compound is an inhibitor of the Aβ-P/Q interaction comprises the steps of
(I) providing the P/Q type voltage-gated presynaptic calcium channel;
(II) additionally providing Aβ(1-42) globulomer and bringing it into contact with the
P/Q type voltage-gated presynaptic calcium channel; and
(III) determining Ca⁺⁺ fluxes through said P/Q type voltage-gated presynaptic calcium channel in the presence and in the absence of the candidate compound; wherein an increase of the Ca⁺⁺ flux through the P/Q type voltage-gated presynaptic calcium channel in the presence relative to the situation in the absence of the candidate compound is indicative of an the candidate compound being an inhibitor of the Aβ-P/Q interaction.

The P/Q type voltage-gated presynaptic calcium channel is known per se (see, e. g., WO98/13490; Qian J and Noebels J L J Neurosci 21: 3721-3728, 2001; Yan Z, et al., 2002, supra). WO98/13490 in particular discloses the cDNA sequence for the human P/Q type voltage-gated presynaptic calcium channel, encoding a protein of 2261 amino acids. Methods for expressing a protein from a cDNA in vertebrate cells are well-documented in the art; e. g. WO96/39512 discloses a process for generating cell lines expressing voltage-gated calcium channels. It is thus within the ken of the skilled person to provide the P/Q type voltage-gated presynaptic calcium channel.

Expediently, the P/Q type voltage-gated presynaptic calcium channel is provided on a living cell, which cell may be either in its natural environment (in situ) or separated therefrom (ex vivo). In a particular embodiment, the cell to be used in the screening method is of a type that naturally expresses the P/Q type voltage-gated presynaptic calcium channel, e. g. a neuronal cell such as a hippocampal neuronal cell. In another embodiment, the cell to be used in the screening method expresses the P/Q type voltage-gated presynaptic calcium channel as a foreign gene. In this embodiment, it is preferred that the cell naturally does not express any other voltage-gated presynaptic calcium channels, e. g. a non-neural cell, e. g. a Xenopus oocyte. Conveniently, expression of the P/Q type voltage-gated presynaptic calcium channel in the cells is verified using standard methology, e. g. by Northern blotting, RT-PCR, Western blotting, cytometry, binding of P/Q-specific ligands such as ω-agatoxin, or pharmacological characterization, i. e. reduction of calcium current after agatoxin application.

In a further particular embodiment, said living cell further comprises an agent for the in situ detection of calcium ion levels (i. e. a calcium sensor agent), e. g. a protein with a calcium-dependent luminescence or fluorescence, such as aequorin or cameleon (Putney P W. Calcium Signaling. CRC Press Inc, 2005). Such calcium sensor agents are well-known to the skilled person, and essentially any of them may be used in the present invention. Without wishing to be bound by theory, it is believed that in suitable agents the conformation of the molecule changes in a manner that depends on the local concentration of Ca⁺⁺, thereby hampering or facilitating physical processes, such as inter- or intramolecular energy transfers, that may be detected and correlated with calcium channel function by the experimentator. Thus, the fluorescence or luminescence of said calcium sensor agents is indicative of the local (e. g. intracellular) calcium levels.

Hence, when the only functional calcium channel of the cell is the P/Q type voltage-gated presynaptic calcium channel, increases in intracellular calcium concentrations

$$( \frac{d [ {Ca}^{++} ]}{d t} > 0 )$$

indicate calcium fluxes through the P/Q type voltage-gated presynaptic dt calcium channel. Therefore, a raise in said increase

$$( \frac{d [ {Ca}^{++} ] c}{d t} > \frac{d [ {Ca}^{++} ] 0}{d t} ,$$

where [Ca⁺⁺]_{c} is the intracellular calcium concentration in the cell in the presence and [Ca⁺⁺]₀ in the absence of the candidate compound) in the presence of Aβ globulomer indicates that a candidate substance is an inhibitor of the Aβ-P/Q interaction and thus potentially useful for the treatment of amyloidoses, as described above.

Suitable methods for the direct determination of ion fluxes, such as the voltage-clamp method, are likewise known in the art (Sakmann B and Neher E. Single-Channel Recording. Springer U S, 97 A.D.). Essentially, conductive microconnections with the in-side and the outside of the cell membrane are established, and the electrical reactivity of the system under different conditions is observed.

The standard method employed here for all determinations of Ca⁺⁺ currents is a patch-clamp method using 120 mM NMG CI₁ 10 mM TEA CI, 14 mM creatine phosphate, 6 mM MgCl₂, 1 mM CaCl₂ 10 mM NMG HEPES, 5 mM Tris₂ ATP and 11 NMG₂ EGTA aS internal, and 30 mM BaCl₂, 100 mM NMG Cl, 10 mM NMG HEPES and 15 mM glucose as external solution, both adjusted to a pH of about 7.2-7.3, for measuring stably transfected BHK (Baby Hamster Kidney) cells expressing the α1 component together with the α2δ and β1B components of the P/Q type voltage-gated presynaptic calcium channel.

Further details of said standard method have been described by Zafir Buraei et al., Roscovitine differentially affects CaV2 and Kv channels by binding to the open state, Neuropharmacology (2006), doi: 10.1016/j.neuropharm.2006.10.006 (corresponds to issue 52, 2007, pages 883-894), which is herein incorporated by reference in its entirety.

Preferably, prior to the measurement irrelevant ion channels are blocked using inhibitors specific for said irrelevant channels (“pharmacological isolation” of the relevant channel or channels), eliminating the dependencies of the electrical status of the membrane on all channels except the one or ones of interest (i.e. the P/Q channel). An inhibitor of the Aβ-P/Q interaction and hence an agent suitable for the treatment of amyloidoses according to the present invention, as mentioned above, will thus be identified as an enhancer of Ca⁺⁺ flux observed in the presence of Aβ when only the P/Q type voltage-gated presynaptic calcium channel is expressed, or when all other calcium channels are blocked.

As all these methods for the determination of Ca⁺⁺ fluxes are essentially quantitative, they are also suitable for the identification of an inhibitor of the Aβ-P/Q interaction with any particularly desired strength of inhibitory effect on the Aβ-P/Q interaction, wherein the strength of the inhibitory effect is the increase in calcium influx induced by the inhibitor in the presence of Aβ globulomer under the conditions selected.

Thus, an agent for the treatment of amyloidoses such as Alzheimer's disease can be identified by determining the effect of said agent on a cell comprising at least the P/Q type voltage-gated presynaptic calcium channel, in particular the effect on the Ca⁺⁺ flux through the P/Q type voltage-gated presynaptic calcium channel of said living cell, in the presence of Aβ globulomer, wherein an inhibitor of the Aβ-P/Q interaction is potentially a suitable agent for the treatment of amyloidoses according to the present invention.

In a particular embodiment of the invention, the inhibitor of the Aβ-P/Q interaction binds to the P/Q type voltage-gated presynaptic calcium channel, preferably with an affinity of K_{D}≦1 μM, more preferably K_{D}≦100 nM, still more preferably K_{D}≦10 nM and most preferably K_{D}≦1 nM, in particular K_{D}<100 pM.

In the context of the present invention, the term “bind” is used generically to denote any immediate physical contact between to molecules, which may be covalent or non-covalent, thus including covalent bonds, hydrogen bridges, ionic interactions, hydrophobic associations, van der Waals forces, etc. It will thus be understood that the term also extends to the temporary association of a first molecule with a catalytically active second molecule, wherein said second molecule performs a modification or modifications on said first molecule which, and consequently whose effects, outlast the actual contact between said first and said second molecule, e. g. generation or removal of covalent bonds.

Suitable methods for determining physical contact between molecules are generally well-known to the person skilled the art and comprise, without being limited to, deter-mining radiation-free energy transfer, radiolabelling of ligands and co-immuno-precipitation.

Alternatively, the inhibitor of the Aβ-P/Q interaction binds to Aβ globulomer, preferably with an affinity of K_{D}<1 μM, more preferably K_{D}≦100 nM, still more preferably K_{D}<10 nM and most preferably K_{D}≦1 nM, in particular K_{D}≦100 pM.

The metabolism of APP and its products such as Aβ is complex and not yet fully understood. Therefore, it is preferred that the inhibitor of the Aβ-P/Q interaction specifically binds to Aβ globulomer, the term “bind specifically to Aβ globulomer” herein being used to denote that the inhibitor shows no significant amount of binding to any other elements of the APP metabolism and in particular no significant amount of binding to the APP protein itself.

The skilled person will understand that an “inhibitor of the Aβ-P/Q interaction” as defined in the present invention may thus bind to the P/Q type voltage-gated presynaptic calcium channel, thereby preventing it, either competitively or by allosteric influences, from participating in the Aβ-P/Q interaction; or to Aβ, in particular to Aβ globulomer, thereby preventing it, either competitively or by allosteric influences, from participating in the Aβ-P/Q interaction.

As used herein, the term “competitive” is used to denote all changes directly influencing a region of intermolecular interaction, which may be covalent or non-covalent, whereas “allosteric” is used to denote all changes not directly influencing a region of intermolecular interaction, which changes may be covalent or non-covalent.

In a preferred embodiment of the invention, the inhibitor reduces the Aβ-P/Q interaction to less than one half of its normal value, preferably to less than one third of its normal value, e. g. to less than 10% of its normal value, wherein the value of the interaction is defined as the difference in activity of the P/Q type voltage-gated presynaptic calcium channel in the presence and in the absence of Aβ globulomer.

According to a further aspect, the invention thus also discloses a pharmaceutical agent or composition for inhibiting the Aβ-P/Q interaction, and its use in the treatment of an amyloidosis such as Alzheimer's disease.

In a particular embodiment of the invention, said agent is an antibody, preferably an anti-P/Q type voltage-gated presynaptic calcium channel antibody, or a fragment or derivative thereof.

As used herein, the anti-P/Q type voltage-gated presynaptic calcium channel antibodies for use in the present invention include polyclonal antibodies (antisera), monoclonal antibodies, recombinant antibodies (including bispecific antibodies), and antigen-binding fragments thereof, e. g. Fab fragments, F(ab′)₂ fragment, and single chain Fv fragments, Fab′ fragments, Fv fragments, and disulfide linked Fv fragments, as well as derivatives thereof. Basically, any antibody, fragment or derivative that binds to the P/Q type voltage-gated presynaptic calcium channel may be used in the present invention. The antibody may be of any class or subclass, e. g. IgM, IgD, IgG, IgA or IgE, and be derived from any commonly used species, e. g. a mammal such as rat, mouse, rabbit, sheep, goat, horse or donkey. Procedures for obtaining suitable antibodies, as well as for fragmenting or derivatizing them, have been described extensively in the art, and are well-known to the skilled artisan. Expediently, a suitable host animal is immunized with the P/Q type voltage-gated presynaptic calcium channel or a fragment or derivative thereof, and the antibodies are isolated in a manner known per se, e. g. using standard hybridoma techniques.

Preferably, the antibody or fragment or derivative thereof does not comprise the portions that are required for induction of biological, in particular immunological, responses; expediently, the Fc part is missing or mutated so not to direct immunological reactions against the P/Q type voltage-gated presynaptic calcium channel. More preferably, the antibody or fragment or derivative thereof is univalent and does not cause cross-linking of the receptors after binding.

For instance, an affinity purified goat polyclonal antibody raised against a peptide mapping near the C-terminus of the α1A subunit of the P/Q type voltage-gated presynaptic calcium channel of human origin is commercially available from Santa Cruz Biotechnology, Inc.

In another particular embodiment of the invention, said agent is an aptamer capable of selectively binding either to the P/Q type voltage-gated presynaptic calcium channel or to Aβ globulomer, the term “aptamer” being used herein to refer to any small molecule that is capable of specific, non-covalent binding to its target, preferably to a peptide, DNA or RNA sequence, more preferably to a peptide, DNA or RNA sequence of about 3 to 100 monomers, in particular of about 5 to 30 monomers, most preferably to a peptide of about 5 to 30 amino acids, which may at one end or both ends be attached to a larger molecule, preferably a larger molecule mediating biochemical functions, more preferably a larger molecule inducing inactivation and/or degradation, most preferably ubiquitin, or preferably a larger molecule facilitating destruction, more preferably an enzyme or a fluorescent protein. Methods for obtaining such aptamers are known per se.

In another particular embodiment of the invention, said agent is a low molecular weight compound, the term “low molecular weight compound” being used herein to refer to a compound with a molecular weight of less than 2000 Da, preferably less than 1000 Da and more preferably less than 500 Da.

In a preferred embodiment of the invention, the inhibitor of the Aβ-P/Q interaction does not exert any inhibitory effect on the P/Q type voltage-gated presynaptic calcium channel when bound.

In a preferred embodiment of the invention, the inhibitor of the Aβ-P/Q interaction does not exert any activating effect on the P/Q type voltage-gated presynaptic calcium channel when bound in the absence of Aβ globulomer.

As used herein, the term “administering” is used to denote delivering an agent to a subject, especially a human subject. Basically, any route of administration known in the art, e. g. buccal, sublingual, oral, rectal, transdermal, subcutaneous, intramuscular, intravenous, intraarterial, intraperitoneal, intrathecal, intralumbaginal or intradural, and any dosage regimen, e. g. as bolus or as continuous supply, may be employed to administer the agent.

The agent may be delivered simply as such or, preferably, in combination with any of a wide range of carriers and excipients, as known in the art, thereby forming a pharmaceutical composition. If desired, a convenient drug targeting and/or delivery system may be used. Expediently, the agent and at least one carrier are combined into a dosage form as known per se to those skilled in the art, e. g. into a controlled or sustained release system. Basically, any carrier and/or excipient compatible with the agent and any kind of dosage form may be used in the methods of the present invention. Suitable compounds and methods are known in the art.

Thus, the present invention will be understood to also relate to the methods and uses relating to the manufacture of pharmaceutical compositions useful in the treatment of amyloidoses. In particular, amyloidoses according to the present invention comprise Alzheimer's disease and Down's syndrome.

In a particular embodiment of the invention, the treatment is a rehabilitating and/or symptomatic treatment.

A “rehabilitating” treatment, as used herein, is, in particular, for providing a benefit with regard to the patient's overall quality of life.

As used herein, a “benefit” is any amelioration in relevant clinical parameters or decrease in subjective suffering of the subject amenable to scoring that can be causally connected to a particular therapeutic measure. Expediently, the benefit is measured by comparing the relevant clinical parameters or the subjective suffering of the subject at a time point before treatment and at least one time point during or after treatment, and expressed in terms of a gain in quality-adjusted life years or disability-adjusted life years (QALYs and DALYs).

The concept of “quality-adjusted life years” and “disability-adjusted life years” is used extensively in the art to evaluate agents and methods, in particular in the context of those diseases where morbidity and disability are medically and socially more of a concern than mortality is, such as dementing diseases. Essentially, each year the life time following treatment is multiplied with an index factor which ranges from 1.0 to indicate perfect quality of life, or zero disability, to 0.0 to indicate death, or complete disability, and the sum of these products is compared to the value obtainable without treatment. Suitable definitions and methods for determining gains and losses in QALYs and DA-LYs, in particular with regard to dementing diseases such as amyloidoses, have been described in the art.

Thus, a benefit is preferably an increase in the aforementioned index factor. In a par-ticular embodiment of the invention, the treatment is hence for providing a benefit to a subject suffering from an amyloidosis.

A “symptomatic” treatment, as used herein, is, in particular, a treatment directed to the abatement or relief of the symptoms of the disease.

In a particular embodiment the present invention relates to a method for the restoration of Aβ-impaired synaptic function and/or plasticity, in particular long-term potentiation, in the subject.

In a further particular embodiment the present invention relates to a method for the restoration of cognitive abilities, memory function and/or performance of activities of daily life (ADL) capacity in the subject.

As used herein, the terms “cognitive abilities”, “synaptic function”, “long-term potentiation” and “memory function” have the meanings as are widely known and used in the art, and their quantificable values are considered as “normal” or “restored” when within the range which is commonly to be expected, e. g. based on long-standing medical practice, appropriate clinical trials and/or biochemical analysis, for the individual subject under consideration when compared to a representative population of other subjects whose essential parameters otherwise agree with those of said subject under consid-eration (peers of said subject). In particular, memory function is considered normal in a subject when said subject upon investigation by suitable means, e. g. short- and/or long-time learning tests, shows no significant deficiencies with regard to memory in function in comparison to a control group matched in species, age, gender and optionally other factors acknowledged as relevant to mental health, which are well-known to those skilled in the art, e. g. blood cholesterol levels, and/or psycho-social factors, e. g. educational and/or occupational background.

As used herein, the term “activities of daily living”, abbreviated “ADL”, is used to denote the essential manual and mental tasks and chores of everyday life, in particular those involving domains of language (impairment thereof being known as “aphasia”), skilled movements (impairment being known as “apraxia” and potentially leading to total loss of control over the body in the final stages of the disease), and the use of cognitive abilities such as recognition (impairment being known as “agnosia”, often accompanied by disorientation and disinhibition, and sometimes also with behavioural changes), and higher-level intellectual functions (such as decision-making and planning). These capacities can be assessed e. g. using questionnaire-based tests well-known in the art, such as the Hodgkinson test (aka. “mini-mental state examination” or MMSE, comprising the recital of basic facts of everyday life) and the Folstein test (aka. “abbreviated mental test score” or AMTS, comprising remembering the time and place of the test, repeating lists of words, arithmetic, language use and comprehension, and copying a simple drawing) for basic mental functions and the John Hopkins Functioning Inventory (aka. JHFI) for basically motoric or movement-related abilities such as sitting, standing, walking, eating, washing, dressing etc.

The skilled person will be aware that in amyloidoses such as Alzheimer's disease the impairment of ADL capacity is dominated, in particular in its early and middle stages, by impairment of the intellectual rather than of motoric or sensory functions, and that even the latter, when found, is due to central rather than peripheral disturbances (e. g. “forgetting how to walk” rather than genuine organic paralysis).

In another aspect the present invention further relates to a method for identifying an inhibitor of the Aβ-P/Q interaction, comprising determining whether a candidate compound exerts an inhibitory effect on the Aβ-P/Q interaction, as disclosed above.

In a particular embodiment of the invention, the method comprises determining the physical contact between Aβ globulomer and the P/Q type voltage-gated presynaptic calcium channel, as disclosed above.

The invention will now be further illustrated by way of reference to the following non-limiting examples and figures. Unless stated otherwise, the terms “A-Beta”, “AR₁₋₄₂”, “Aβ”, “αβ”, “glob” all denote the Aβ(1-42) globulomer described in reference example 2. “Kontrolle” means “control”.

### BRIEF DESCRIPTION OF THE FIGURES

Effect of Aβ(1-42) globulomer on spontaneous synaptic activity as recorded from rat primary cultured hippocampal neurons by voltage clamp:

FIG. 1A and FIG. 1C are recordings of spontaneously occurring synaptic currents in a cultured hippocampal neuron (downward deflections indicate the postsynaptic currents which are elicited by neurotransmitter release from one or more presynaptic neurons; application of the globulomer and washout (top trace) are indicated); FIG. 1B and FIG. 1D are the cumulative probability functions.

FIG. 2: Effect of Aβ(1-42) globulomer on the frequency of synaptic currents.

FIG. 3: Effect of Aβ(1-42) globulomer on the frequency of mIPSCs in of cells cultivated with 0.5 μM ω-conotoxin MVIIA to achieve synaptic P/Q predominance (n=6): Number of synaptic events during 5 min relative to non-Aβ globulomer treated cells. Left to right: (1) non-Aβ globulomer treated P/Q-dominated cells=reference, (2) P/Q-dominated cells treated with Aβ globulomer (at a concentration corresponding to approximately 1 μM of Aβ monomer).

FIG. 4: Aβ(1-42) globulomer has no effect on the amplitude of mIPSCs of cells cultivated with ω-conotoxin MVIIA to achieve synaptic P/Q predominance: Average amplitude of synaptic events relative to non-Aβ globulomer treated cells. Left to right: (1) non-Aβ globulomer treated P/Q-dominated cells=reference, (2) P/Q-dominated cells treated with Aβ globulomer (at a concentration corresponding to approximately 1 μM of Aβ monomer).

FIG. 5: Effect of ω-agatoxin on the frequency of mlPSCs in of cells cultivated with 0.5 μM ω-conotoxin MVIIA to achieve synaptic P/Q predominance (n=3):

Number of synaptic events during 5 min relative to non-ω-agatoxin treated cells. Left to right: (1) non-ω-agatoxin treated P/Q-dominated cells=reference, (2) P/Q-dominated cells treated with 0.5 μM ω-agatoxin.

FIG. 6: No additive effect on the frequency of mIPSCs in of cells cultivated with 0.5 μM ω-conotoxin MVIIA to achieve synaptic P/Q predominance after blockade of P/Q-channels by ω-agatoxin (n=6): Number of synaptic events during 5 min relative to non-Aβ globulomer treated cells. Left to right: (1) non-Aβ globulomer treated P/Q-dominated cells (ω-agatoxin only)=reference, (2) P/Q-dominated cells treated with Aβ globulomer (at a concentration corresponding to approximately 1 μM of Aβ monomer) after pre-treatment with 0.5 μM ω-agatoxin.

FIG. 7: No effect of globulomer on the amplitude of mIPSCs when P/Q channels of P/Q-dominated cells are already blocked by 0.5 μM ω-agatoxin IVA (n=6): Number of synaptic events during 5 min relative to non-Aβ globulomer treated cells. Left to right: (1) non-Aβ globulomer treated P/Q-dominated cells (ω-agatoxin only)=reference, (2) P/Q-dominated cells treated with Aβ globulomer (at a concentration corresponding to approximately 1 μM of Aβ monomer) after pre-treatment with 0.5 μM ω-agatoxin.

FIG. 8: Agatoxin does not impair spontaneous synaptic activity in cultures that lack functional P/Q-type Ca⁺⁺ channels: Number of synaptic events during 5 min was set to 100% for each cell analysed. The right bar indicates the relative number of synaptic events in each cell after application of 0.5 μM ω-agatoxin.

FIG. 9: Globulomer does not impair spontaneous synaptic activity in cultures that lack functional P/Q-type Ca⁺⁺ channels: Number of synaptic events during 5 min relative to non-Aβ globulomer treated cells was set to 100% for each cell analysed. The right bar indicates the relative number of synaptic events in each cell after application of Aβ globulomer (at a concentration corresponding to approximately 1 μM of Aβ monomer).

FIG. 10: Suppression of spontaneous synaptic currents by Aβ(1-42) globulomer and its reversal by the P/Q channel agonist roscovitine: Number of synaptic events during 5 min relative to non-Aβ globulomer treated P/Q-dominated cells. Left to right: (1) non-Aβ globulomer treated P/Q-dominated same cells=reference, (2) P/Q-dominated same cells treated with Aβ globulomer (at a concentration corresponding to approximately 1 μM of Aβ monomer), (3) P/Q-dominated same cells treated simultaneously with Aβ globulomer (at a concentration corresponding to approximately 1 μM of Aβ monomer) and 20 μM roscovitine.

FIG. 11: No effect on the amplitude of spontaneous synaptic currents of the P/Q channel agonist roscovitine: Average amplitude of synaptic events relative to non-Aβ globulomer treated P/Q-dominated cells. Left to right: (1) non-Aβ globulomer treated P/Q-dominated same cells=reference, (2) P/Q-dominated same cells treated with Aβ globulomer (at a concentration corresponding to approximately 1 μM of Aβ monomer), (3) P/Q-dominated same cells treated simultaneously with Aβ globulomer (at a concentration corresponding to approximately 1 μM of Aβ monomer) and 20 μM roscovitine.

FIG. 12: The effect of Aβ(1-42) globulomer on spontaneous synaptic activity of P/Q-dominated cells can be reversed by the P/Q channel agonist roscovitine: Synaptic potentials over time. Arrows indicate the time points when Aβ globulomer (at a concentration corresponding to approximately 1 μM of Aβ monomer) and 20 μM roscovitine, respectively, were added.

FIG. 13: Reducing effect of Aβ globulomer on the amplitude of pharmacologically isolated P/Q-type calcium channels: Traces represent membrane currents after activation of P/Q-type channels by a depolarizing voltage step. Left to right: (1) P/Q-current under control conditions, (2) P/Q-current of the same cell after application of Aβ globulomer (at a concentration corresponding to approximately 1 μM of Aβ monomer), (3) P/Q-current of the same cell after washout of Aβ globulomer.

FIG. 14: Effect of Aβ(1-42) globulomer on the pharmacologically isolated P/Q current at different time points: Average amplitude of P/Q-mediated current amplitude relative to non-Aβ globulomer treated P/Q-dominated cells. Left to right: (1) non-Aβ globulomer treated same cells=reference, (2) same cells 10 min after treatment with Aβ globulomer (at a concentration corresponding to approximately 1 μM of Aβ monomer), (3) same cells 15 min after treatment with Aβ globulomer (at a concentration corresponding to approximately 1 μM of Aβ monomer).

FIG. 15: Effect of 0.5 μM ω-agatoxin IVA on the pharmacologically isolated P/Q current at different time points: Average amplitude of P/Q currents relative to non-ω-agatoxin treated P/Q-dominated same cells. Left to right: (1) non-ω-agatoxin treated P/Q-dominated same cells=reference, (2) P/Q-dominated cells 10 min after treatment with 0.5 μM ω-agatoxin, (3) P/Q-dominated cells 15 min after treatment with 0.5 μM ω-agatoxin.

FIG. 16: Effect of Aβ on the pharmacologically isolated P/Q current at different time points, revealing the effect of washing out the Aβ globulomer: Average amplitude of P/Q-mediated current relative to non-Aβ globulomer treated P/Q-dominated cells. Left to right: (1) non-Aβ globulomer treated P/Q-dominated cells=reference, (2) P/Q-dominated cells 10 min after treatment with 83 nM Aβ globulomer (at a concentration corresponding to approximately 1 μM of Aβ monomer), (3) P/Q-dominated cells 15 min after treatment with Aβ globulomer (at a concentration corresponding to approximately 1 μM of Aβ monomer), (4) P/Q-dominated cells treated with Aβ globulomer (at a con-centration corresponding to approximately 1 μM of Aβ monomer) after washing out the Aβ globulomer.

FIG. 17: Effect of Aβ on spontaneous synaptic activity in the hippocampal slice: Number of synaptic events during 5 min relative to non-Aβ globulomer treated tissue. Left to right: (1) non-Aβ globulomer treated same slice=reference, (2) same slice treated with Aβ globulomer (at a concentration corresponding to approximately 1 μM of Aβ monomer).

FIG. 18: Results of affinity approach with immobilized Aβ(1-42) globulomers

The SeeBlue Prestined Marker is represented with M. A represents the 80,000 g membrane-protein fraction, and B was used for the 150,000 g residual membrane protein fraction. The gels were loaded in the following order.

A1: 5 μg of membrane proteins before affinity chromatography

A2: Unbound proteins after affinity chromatography

A3: PBS/0.5% SDS elution

B1: 5 μg of residual membrane proteins before affinity chromatography

B2: Unbound proteins after affinity chromatography

B3: PBS/0.5% SDS elution

FIG. 19: Spontaneous synaptic activity is reversibly suppressed by Aβ(1-42) globulomer. Original recording of spontaneously occurring synaptic currents in a cultured hippocampal neuron before (top), during (middle) and after (bottom) application of Aβ(1-42) globulomer.

Effects of Aβ(1-42) globulomer on different types of synaptic currents in cultured hippocampal neurons. White bars: effect of Aβ(1-42) globulomer; black bars: washout for at least 10 min.

FIG. 20A: Reduction of event frequency as percentage of previously recorded control currents (1.0). FIG. 20B: Effects of Aβ(1-42) globulomer on median amplitude of the respective currents. sPSCs: spontaneously occurring pharmacologically naive postsynaptic currents; mPSCs: pharmacologically naive miniature postsynaptic currents recorded in the presence of TTX; mIPSCs: miniature inhibitory postsynaptic currents; sEPSCs: spontaneously occurring excitatory postsynaptic currents; mEPSCs: miniature excitatory postsynaptic currents.

Stability of GABA_{A} receptor-mediated currents towards Aβ(1-42) globulomer.

FIG. 21A: Repetitive application of 100 μM GABA to a cultured hippocampal neuron yields stable inward current before, during, and after application of the oligomer. FIG. 21B: Enlarged view of current traces marked with * in FIG. 21A. Note the stability of response in the absence (left) and presence (right) of Aβ(1-42) globulomer. FIG. 21C: Time course of GABA-induced currents from 5 cells recorded in control solution (dashed line) and from 3 neurons where Aβ(1-42) globulomer was applied (continuous line, time of application indicated by bar). Amplitudes normalized to the last GABA-induced current before application of Aβ(1-42) globulomer.

Suppression of P/Q-type calcium currents by Aβ(1-42) globulomer.

FIG. 22A: Time course of current amplitudes upon application of globulomer. Currents were elicited by voltage steps to −10 mV. FIG. 22B: Example traces of P/Q-type currents before, during and after globulomer.

Steady-state activation and inactivation parameters of P/Q currents.

FIG. 23A: Current/voltage relationship before globulomer (squares) and during Aβ(1-42) (triangles). A reduction of the current amplitudes over the entire voltage-range, were the current could be activated, was observed following application of the globulomer. FIG. 23B & FIG. 23C: No difference in steady-state activation (B) and inactivation curves (C) for P/Q channel-mediated barium currents in the absence and presence of Aβ(1-42) globulomer. FIG. 23D: A significant decrease in maximal conductance (gₘₐₓ) of the P/Q channels was induced by Aβ(1-42) globulomer.

Pharmacological modulation of the effect of Aβ(1-42) globulomer by agents interacting with P/Q-type calcium channels.

FIG. 24A: Effects of Aβ(1-42) globulomer on frequency of mixed synaptic currents. FIG. 24B: Effects on median ampli-tude. Values are given relative to data in control solution. Note suppression of the effect by ω-agatoxin and partial recovery of event frequency by roscovitine.

FIG. 25 Enhancing P/Q calcium currents by roscovitine prevents/reverses chronic Aβ globulomer-induced deficits on evoked synaptic transmission in hippocampal tissue (slice cultures). Recordings were performed after incubation with Aβ(1-42) globulomer (at a concentration corresponding to approximately 1 μM of Aβ monomer), Aβ(1-42) globulomer (at a concentration corresponding to approximately 1 μM of Aβ monomer)+20 μM roscovitine, or control (SDS).

Effect of extracellular Ca²⁺ on sPSC frequency after treatment with Aβ(1-42) globulomer.

FIG. 26A: Original recording of sPSCs before (control in 1 mM Ca²⁺), after addition of Aβ(1-42) globulomer (glob in 1 mM Ca²⁺) and after subsequent elevation of Ca²⁺-concentration (glob in 4 mM Ca²⁺). FIG. 26B: Reduction of event frequency after application of Aβ(1-42) globulomer (p<0.05; n=6) and partial recovery after elevation of Ca²⁺ from 1 mM to 4 mM. Values are given as percentage of control currents. FIG. 26C: Event frequency of single cells (n=6) after application of Aβ(1-42) globulomer and after subsequent elevation of Ca²⁺ from 1 mM to 4 mM. Values are given as percentage of control currents. FIG. 26D: No difference in median amplitude after application of Aβ(1-42) globulomer (n=6) and after subsequent elevation of Ca²⁺. Values are given as percentage of control currents. FIG. 26E: Original recordings of massive discharges directly after Ca²⁺ elevation for the cell shown in FIG. 26A. These currents were rejected from analysis.

FIG. 27 Inhibiting the interaction of Aβ(1-42) globulomer with the P/Q calcium channels by anti-P/Q type voltage-gated presynaptic calcium channel antibody prevents chronic Aβ globulomer-induced deficits on evoked synaptic transmission in hippocampal tissue. Recordings were performed after incubation with Aβ(1-42) globulomer (at a concentration corresponding to approximately 1 μM of Aβ monomer), Aβ(1-42) globulomer (at a concentration corresponding to approximately 1 μM of Aβ monomer)+0.3 μg/ml (=approximately 2 nM) anti-P/Q antibody, or control (SDS).

FIG. 28 Bar diagram showing no effect of the monomer on mPSC frequency compared with the significant reduction in frequency induced by the globulomer. The right bar shows that the solvent alone (0.0001% NaOH) does not affect the frequency.

### First Series of Experiments

### Reference Example 1

### Determination of Synaptic Potentials

Neuronal cells from the rat hippocampus were obtained and cultured in accordance with methods known per se in the art (Banker G A, Cowan W M, Brain Res. 1977 May 13; 126(3):397-42). Cultured neurons show spontaneous postsynaptic currents (PSCs), i. e. spontaneous PSCs and, in the presence of the sodium channel blocker tetrodotoxin, miniature PSCs. As mentioned above, the influx of Ca⁺⁺ through presynaptic ion channels such as the N, P/Q and R type voltage-gated presynaptic calcium channels is what causes the release of neurotransmitter from preformed vesicles in presynaptic terminals. The measured signal reflects the current response of the postsynaptic cell to the release of such transmitters, e.g. gamma-aminobutyric acid or glutamate.

For measurements, primary cell cultures were transferred to a recording chamber mounted on a microscope (Olympus CKX1) and were immersed at room temperature into a buffered solution consisting of 156 mM NaCl, 2 mM KCl, 2 mM CaCl₂, 1 mM MgCl₂, 16.5 mM glucose and 10 mM HEPES at a pH of 7.3. The osmolarity of the solution was 330 mosmol.

Electrodes were produced by pulling from borosilicate capillaries (available from Science Products) with a horizontal pipette pulling device (P-97 from Sutter Instruments). After filling with the intracellular solution, the final resistance of the electrodes was from 2 to 5 MΩ. The intracellular solution consisted of either (for recordings of miniature PSCs) 100 mM KCl, 10 mM NaCl, 0.25 mM CaCl₂, 5 mM EGTA, 40 mM glucose, 4 mM MgATP and 0.1 mM NaGTP at a pH of 7.3, or (for recording of calcium currents) 1 10 mM CsCl, 10 mM EGTA, 25 mM HEPES, 10 mM tris-phosphocreatine, 20 U/ml creatine phosphokinase, 4 mM MgATP and 0.3 mM NaGTP.

All test compounds were applied either by bath perfusion or by addition to the bath by means of a micropump connected to a manually guided pipette.

All recordings of miniature PSCs were made in the presence of 0.5 μM tetrodotoxin (TTX; available from Tocris Bioscience) to block the Na⁺ and K⁺ channels in the neuronal cell membrane which would otherwise also influence the electrical status of the membrane. For calcium current recordings the extracellular solution contained 140 mM TEA-CI (to block K⁺-channels) 10 mM BaCl₂, 0.5 μM TTX, 10 mM HEPES and 20 mM glucose at a pH 7.3. When required, ω-conotoxin MVIIA (available from Alomone Labs, Jerusalem, Israel) was added to a final concentration of 0.5 μM to block N type voltage-gated presynaptic Ca⁺⁺ channels, thereby “pharmacologically isolating” the ion fluxes through the P/Q type voltage-gated presynaptic calcium channel. If necessary, L-type voltage-gated calcium channels were blocked by addition of 10 μM nifedipine.

To mimic the effect of Aβ globulomer as P/Q type blocker, ω-agatoxin IVA (available from Alomone Labs, Jerusalem, Israel) was added to a final concentration of 0.5 μM to specifically block the P/Q type voltage-gated presynaptic Ca⁺⁺ channels of the sample cell.

All substances were stored as lyophilized powders at −20° C. Stock solutions were prepared with vehicles appropriate for the solubility (i. e. immersion solution). Vehicle was destilled water or standard extracellular solution for all drugs except nifedipine, which was dissolved in ethanol, and roscovitine, which was dissolved in dimethyl sulfoxide (DMSO). The final concentration of the solvents in the Aβ-globulomer solvent buffer which was applied to neurons was <1%/o and the final concentration of DMSO was <1.5% o.

Whole-cell patch-clamp recordings (sPSCs and mPSCs) were conducted in a manner essentially known per se (see, e.g., Sakmann B and Neher E. Single-Channel Recording. Springer U S, 97 A.D.) at a holding potential of −70 mV using an EPC7 amplifier (available from HEKA Electronics). Signals were filtered at 3 kHz and sampled at 20 kHz.

After formation of a seal, rupture of the membrane by the electrode and establishment of the whole-cell configuration, the perfusion of the bath was stopped, and the substances to be tested were injected into the bath using a custom-made syringe pump.

The sPSCs or mPSCs were then recorded for 10 minutes giving the control values before any toxins were added.

For the selective determination of P/Q type voltage-gated presynaptic calcium channel currents, the cells were activated in a manner known per se (see Yan et al., 2002, supra) by a voltage protocol, where the cells were excited by depolarization to −10 mV for 50 ms every 20 sec. After the formation of the whole-cell configuration, currents increased steadily until they had reached a stable amplitude level. After this stable amplitude level had been established, the effects of different test compounds on the rate of ion flux were observed and expressed in terms of the normalized mean P/Q amplitude and standard error of the mean SEM. Frequency and amplitude of synaptic currents were calculated offline using a template-based algorithm (custom made routine within the Signal and Spike software, purchased from CED Inc., Cambridge, UK).

When desired, the measurement was evaluated at several timepoints and optionally after a washout. Student's t-test was applied to determine significance, p<0.05 being considered as indicative of significant differences.

### Reference Example 2

### Generation of Aβ Globulomer

An Aβ(1-42) globulomer preparation with an apparent molecular weight of 38/48 kDa as determined by SDS-PAGE was obtained as described in Example 6b of WO2004/067561. Essentially, Aβ monomer was pretreated with HFIP for dissolving hydrogen bonds, then diluted and further incubated in the presence of 0.2% SDS₁ fol-lowed by isolation of the thus formed globulomer.

In brief, lyophilized Aβ(1-42) synthetic peptide was disaggregated by using 100% 1,1,1,3,3,3 hexafluoro-2-propanol. After evaporation, Aβ(1-42) was resuspended at a concentration of 5 mM in dimethylsulfoxide, diluted to a final concentration of 400 μM in PBS containing 0.2% SDS. After 6 h incubation at 37° C., the sample was diluted with three volumes of H₂O and incubated for another 18 h at 37° C. The sample was concentrated by ultrafiltration (30 kDa cutoff), dialyzed against 5 mM NaH₂PO₄ 35 mM NaCl, pH 7.4, centrifuged at 10,000×g for 10 min, and the supernatant containing the 48 kDa Aβ(1-42) globulomer withdrawn. Aβ(1-42) globulomer was diluted in extracellular solution at the concentration indicated immediately before experiments. Currents were measured before and immediately after addition of Aβ(1-42) globulomer to the bath solution.

For control experiments, synthetic monomeric Aβ(1-42) peptide (H-1368; Bachem, Bubendorf, Switzerland) was dissolved in 0.1% NaOH, yielding a 1 mM stock solution, which was frozen at −80° C. Immediately before the experiment, this solution was dissolved at 1:500 in bath solution, which was added to the bath by means of a micro-pump, resulting in a final concentration of 1 μM.

### Example 3

### Inhibitory Effect of Aβ Globulomer on Spontaneous Synaptic Activity

Using acute application of the P/Q channel blocker ω-agatoxin as a negative control and cells untreated with regard to the P/Q type voltage-gated presynaptic calcium channel as a positive control, the effects of Aβ(1-42) globulomer on the frequency of spontaneous synaptic events in cultured hippocampal neurons treated with ω-cono-toxin to achieve synaptic dominance of the P/Q type channel, as described in Reference Example 1, were observed.

Aβ globulomer, obtained as described in Reference Example 2, was tested according to the procedure described in Reference Example 1 for channel function inhibitors such as ω-agatoxin. In the presence of ω-agatoxin, Aβ globulomer had no further effect on synaptic activity, indicating that the effects of both agents involved a common mechanism. A total of 200 μl Aβ-globulomer solvent buffer comprising a Aβ(1-42) globulomer concentration corresponding to approximately 2 μM of Aβ monomer was added to the bath (previous volume 200 μl), resulting in a final Aβ(1-42) globulomer concentration corresponding to approximately 1 μM of Aβ monomer. Based on the assumption that the Aβ(1-42) globulomer consists of 12 Aβ(1-42) monomers a final Aβ(1-42) globulomer concentration of approximately 83 nM can be calculated. Measurements of synaptic activity were then taken.

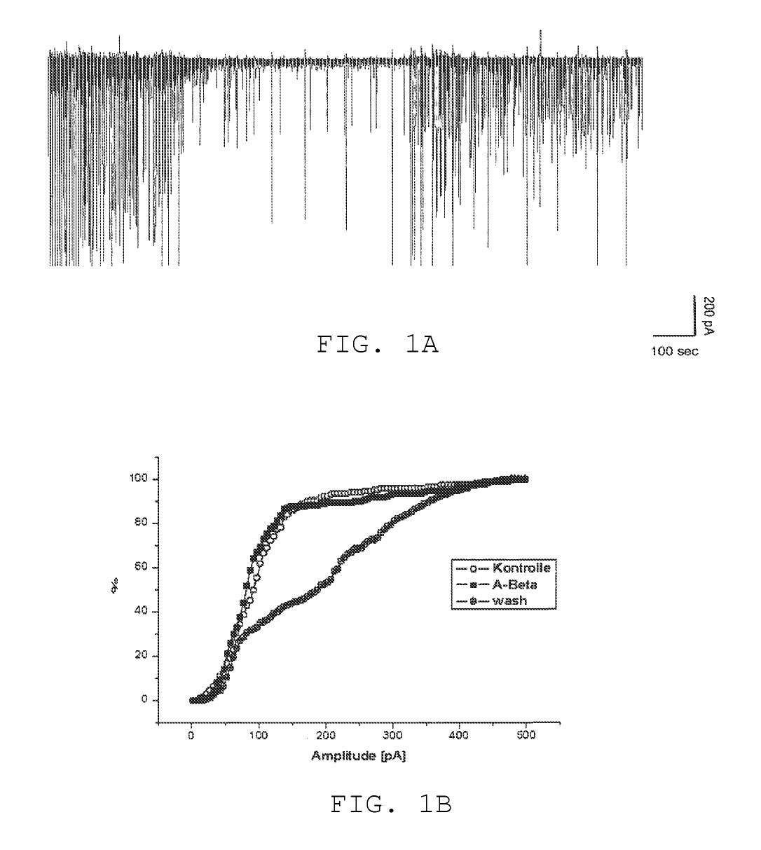

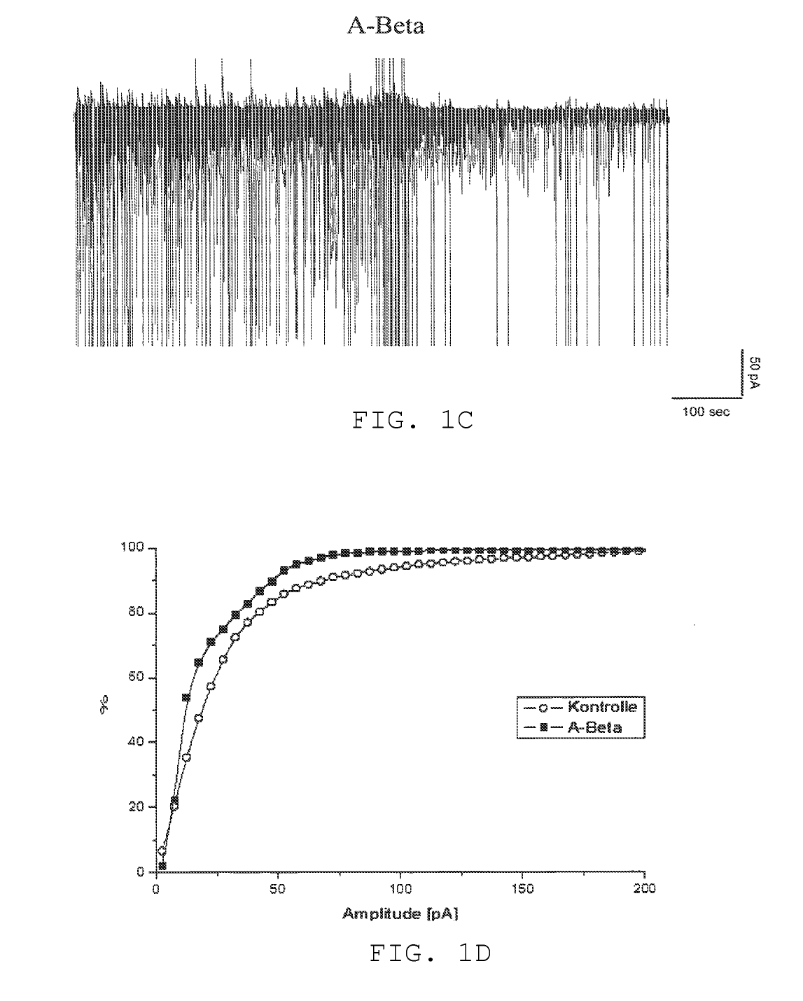

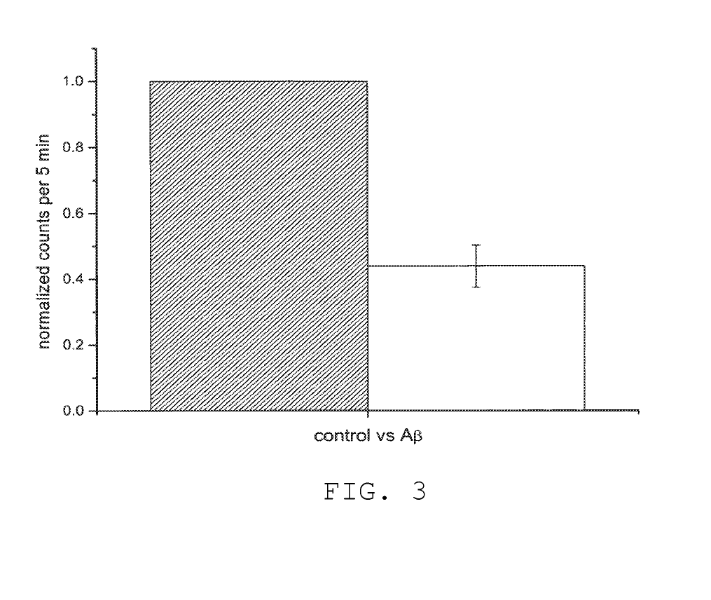

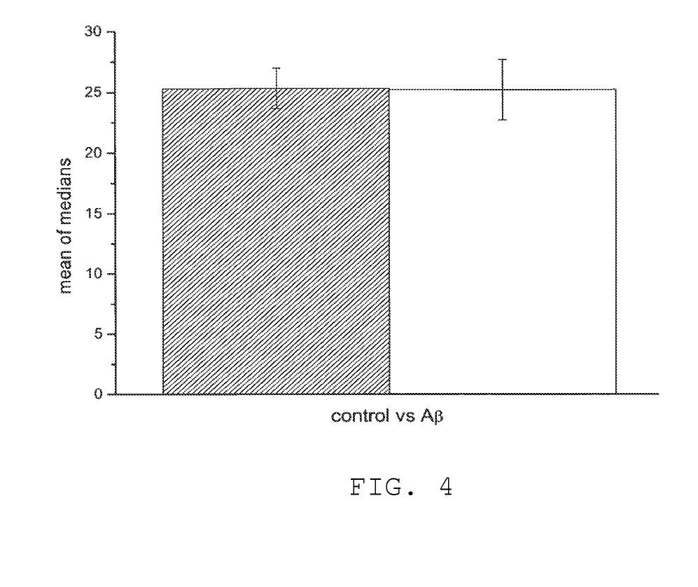

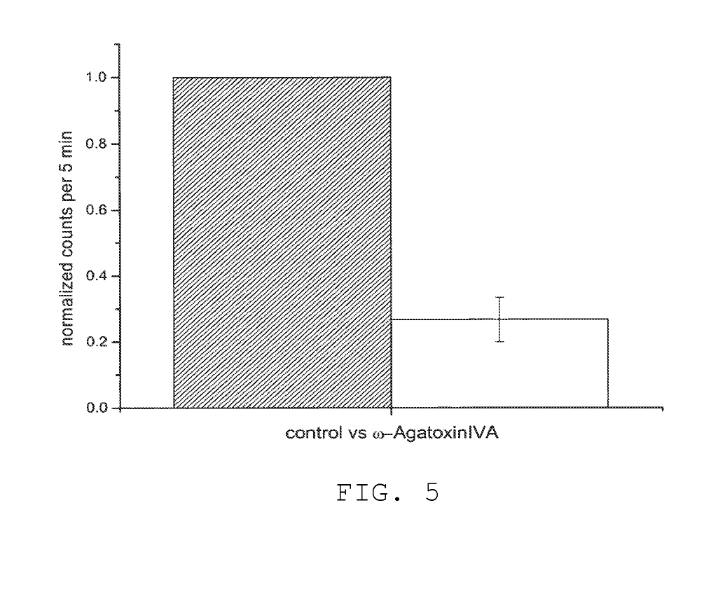

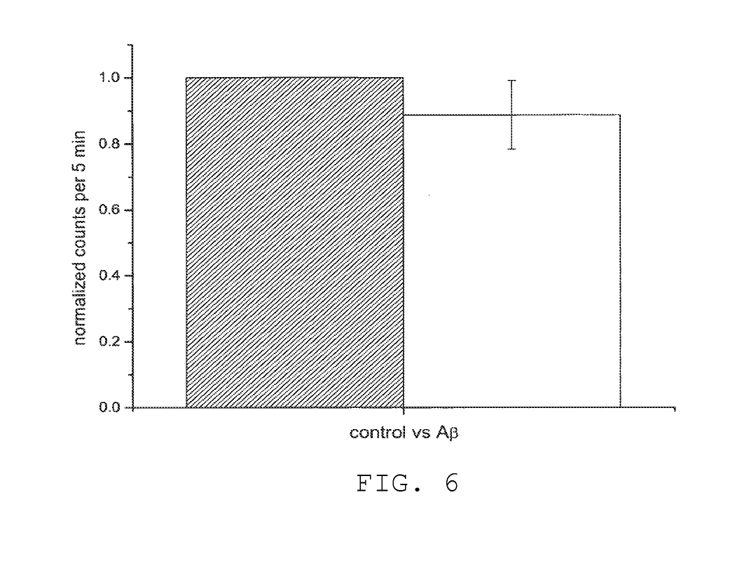

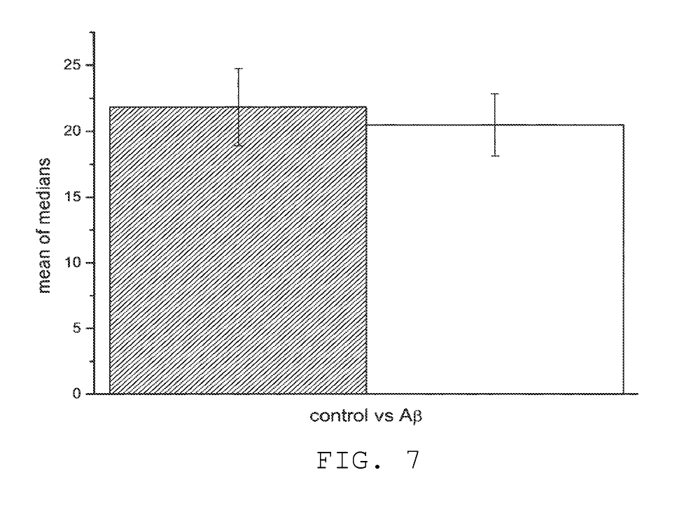

Results are shown in FIGS. 1-7, demonstrating that the Aβ globulomer inhibits the frequency of spontaneous synaptic events with an efficiency approaching that of the strong P/Q inhibitor ω-agatoxin but has no or little effect on the amplitude of the synaptic events. Thus, Aβ(1-42) globulomer reduces synaptic activity, most likely by a presynaptic mechanism, which shares crucial elements with the effect of ω-agatoxin.

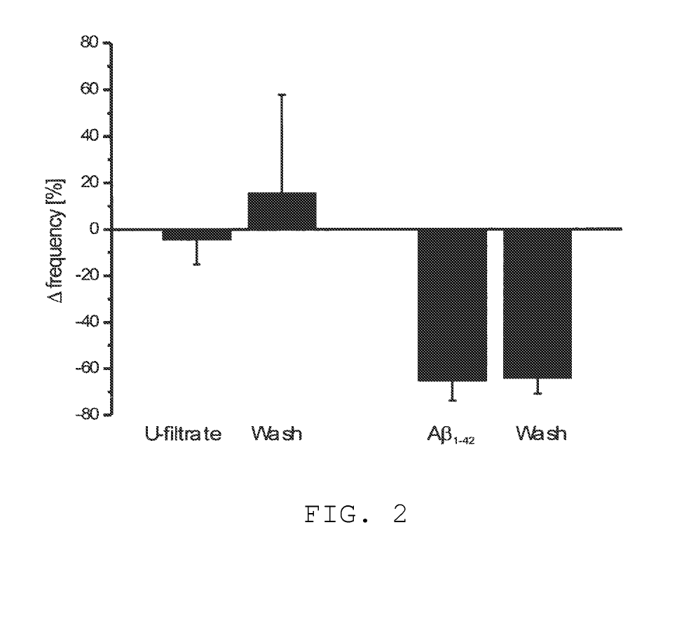

These results were verified by subjecting the Aβ(1-42) globulomer containing Aβ-globulomer solvent buffer to ultrafiltration with a filter having a molecular cutoff size of 5 kDa for globular proteins. The resulting solvent buffer contained no detectable amounts of Aβ globulomer protein prior to bringing it into contact with the cells. The ultrafiltrate had no effect on the synaptic events (see FIG. 2), indicating that the agent responsible for reducing the frequency of spontaneous synaptic events was unable to pass ultrafilters.

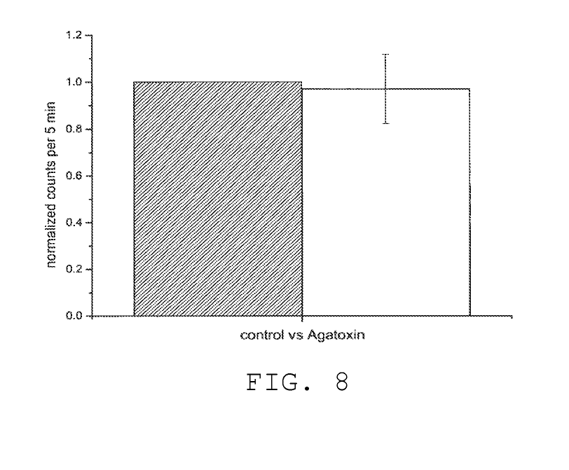

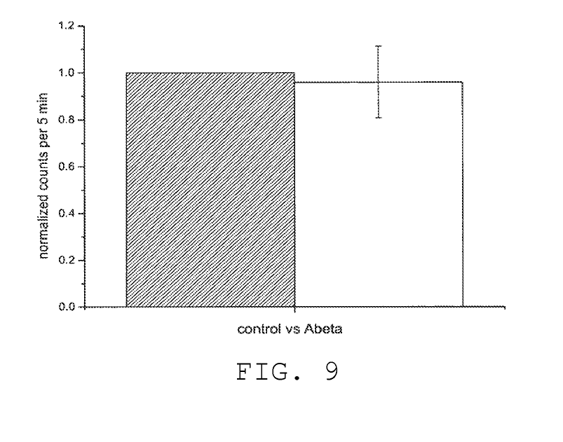

Furthermore, the effect of Aβ(1-42) globulomer is absent in cells predominantly expressing presynaptic N-type calcium channels. Results are shown in FIGS. 8 and 9, demonstrating that in the N-dominated cells no reduction of the frequency nor any reduction in amplitude is achieved by either ω-agatoxin or Aβ globulomer, i. e. that both ω-agatoxin and Aβ globulomer target the P/Q type voltage-gated presynaptic calcium channel.

### Example 4

### Rescue of Spontaneous Synaptic Activity by Roscovitine

Using the Aβ(1-42) globulomer of Reference Example 2 as a negative control and cells untreated with regard to the P/Q type voltage-gated presynaptic calcium channel as a positive control, the effects of the P/Q type voltage-gated presynaptic calcium channel activator roscovitine on the Aβ globulomer-induced reduction of the frequency of spontaneous synaptic events in cultured hippocampal neurons treated with ω-conotoxin, as described in Reference Example 1, were observed.

Roscovitine was used at a final concentration of 20 μM, by adding it simultaneously with Aβ(1-42) globulomer (final concentration of Aβ globulomer corresponding to approximately 1 μM of Aβ monomer). Roscovitine is known (Zhen Yan et al., J. Physiol. 540: 761-770 (2002)) to slow down the inactivation of the P/Q type voltage-gated presynaptic calcium channel, i. e. to extend the time for which a channel, once opened, remains in the open state, thereby increasing the calcium ion flow through the P/Q type voltage-gated presynaptic calcium channel.

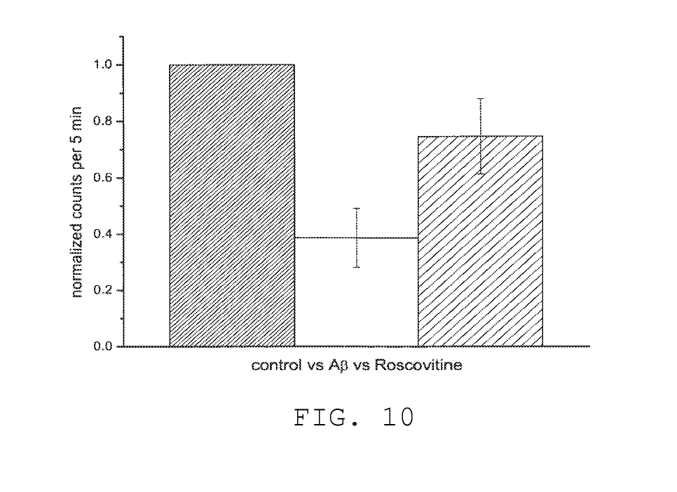

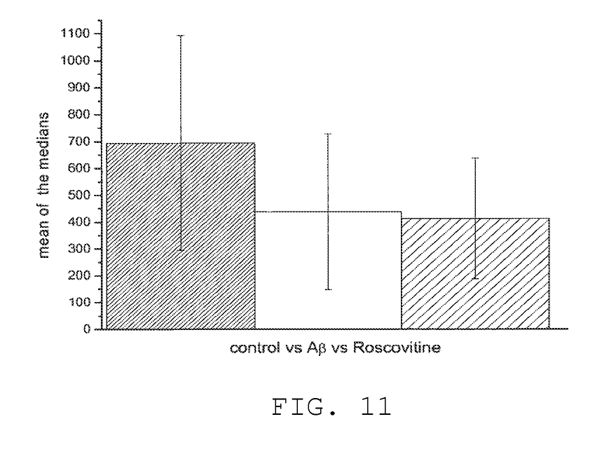

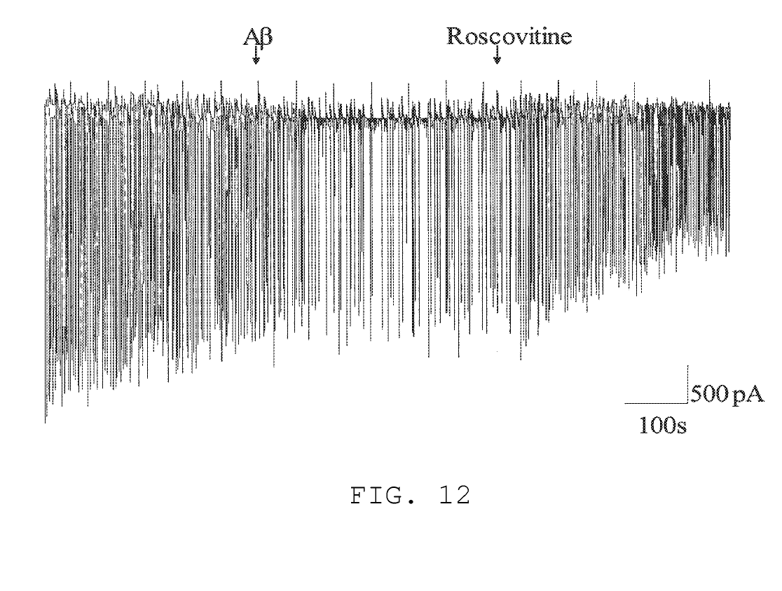

Results are shown in FIGS. 10 and 11, demonstrating that a P/Q type voltage-gated pre-synaptic calcium channel activator is capable of restoring the frequency of spontaneous synaptic events under the influence of Aβ globulomer to almost that of untreated cells, i. e., that a P/Q activator may be used to reverse the detrimental effects of Aβ globulomer.

### Reference Example 5

### Direct Determination of the Activity of the P/Q Type Voltage-Gated Presynaptic Calcium Channel, and of Inhibitory and Activating Influences, by the Voltage-Clamp Method

Cells were prepared and subjected to measurement of membrane currents by the voltage-clamp method basically as described in Reference Example 1, the difference being essentially that all irrelevant (non-P/Q type) ion channels of the cells were blocked chemically, thereby allowing for direct determination of the ion fluxes rather than of the resulting IPSCs. Blocking of the irrelevant channels was achieved using the following additions to the bath or electrode solution:

| Compound | Concentration | | Channel blocked |
|---|---|---|---|
| TEA-Cl | 140 | mM | I[K⁺] |
| BaCl₂ | 10 | mM | |
| CsCl (in the pipette) | 110 | mM | |
| Nifedipine | 10 | mM | L-type Ca⁺⁺ channel |
| ω-conotoxin MVIIA | 0.5 | mM | N-type Ca⁺⁺ channel |

| | | |
|---|---|---|
| Tetrodotoxin | 0.5 | Na⁺ channels |

The Ba⁺⁺ also served as the charge carrier (i. e. substrate replacement) for the P/Q type voltage-gated presynaptic Ca⁺⁺ channel, with the additional advantage that conductance through this channel and hence the sensitivity of the assay were thereby increased to approximately tenfold. This made it possible to directly detect ion fluxes through P/Q-channels in somatic recordings.

In order to prevent the “run down” of Ca⁺⁺ currents in the samples, the electrode solution also comprised, in addition to the substances listed above, 10 mM tris-phospho-creatinine and 20 U/ml creatine phosphokinase, which together served as an ATP re-generating system preventing “run-down”, i.e. decline due to a gradual loss of channel conductance, of the observed currents. ATP is needed to maintain the conductance of the calcium channels over time intervals longer than several minutes, allowing to conduct the described pharmacological experiments with sufficiently stable calcium currents.

### Example 6

### Direct Effect of Aβ Globulomer on the P/Q Type Voltage-Gated Presynaptic Calcium Channel in Cultured Cells

Using ω-agatoxin as a negative control and cells untreated with regard to the P/Q type voltage-gated presynaptic calcium channel as a positive control, the effects of approximately Aβ(1-42) globulomer of Reference Example 2 (at a concentration corresponding to approximately 1 μM of Aβ(1-42) monomers) on calcium flux in hippocampal neurons treated with ω-conotoxin were directly observed as described in Reference Example 5.

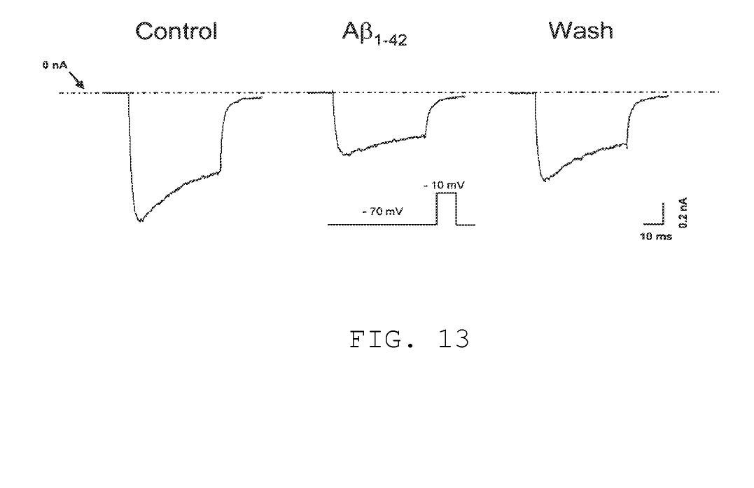

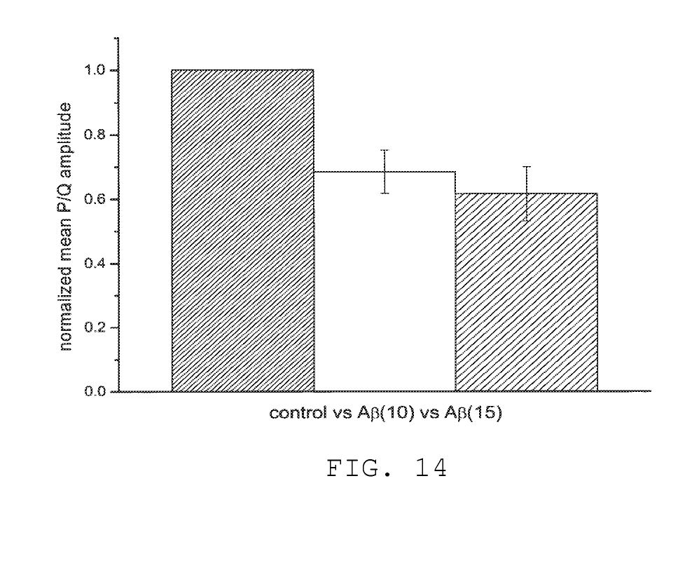

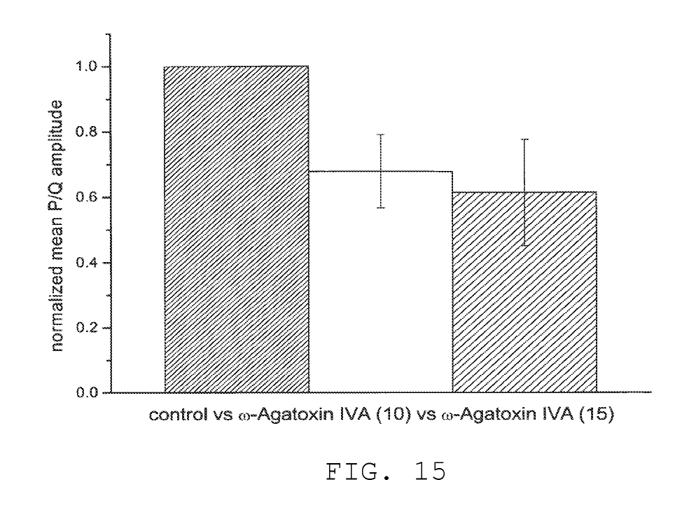

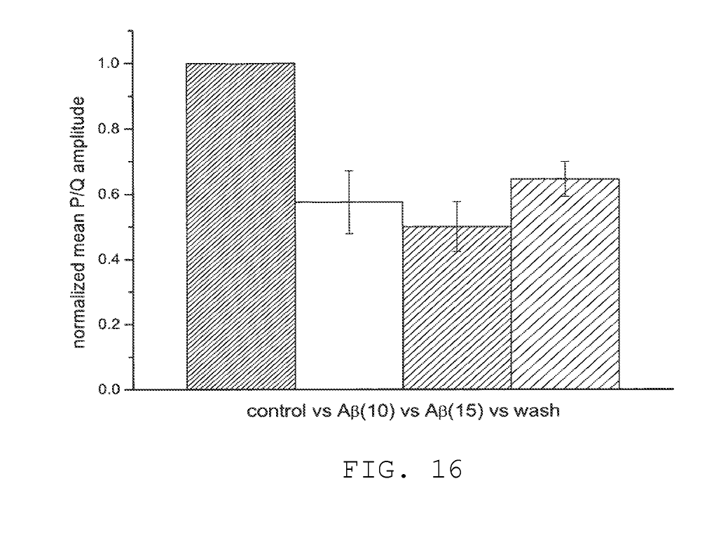

Recordings were taken at 10 min and 15 min and optionally after a washout. Typical results are shown in FIGS. 13-16. These findings demonstrate that Aβ globulomer directly inhibits the activity of the P/Q type voltage-gated presynaptic calcium channel and cannot be readily washed out after binding to the P/Q type voltage-gated presynaptic calcium channel. They further demonstrate that Aβ globulomer impedes, by decreasing the amplitude of the calcium flux, the initiation of synaptic currents.

### Example 7

### Direct Effect of Aβ Globulomer on the P/Q Type Voltage-Gated Presynaptic Calcium Channel In Situ

To verify whether the effect of the globulomer on neurons in cell cultures also takes place in the more organotypic slice-preparation of the hippocampus, synaptic currents were determined in this tissue.

400 μm thick slices were prepared from freshly dissected hippocampi of the mouse using a method known per se (Dingledine R. Brain Slices. New York: Plenum Press, 1983). CA1 pyramidal cells were patched and spontaneous synoptical currents were recorded prior and after application of Aβ(1-42) globulomer via an Eppendorff pipette.

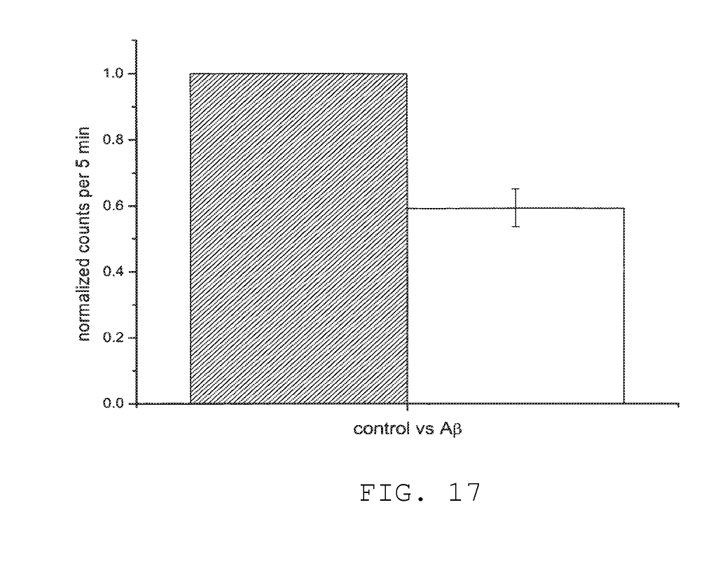

Typical results are shown in FIG. 17. These findings demonstrate that the mechanism for Aβ globulomer mediated inhibition disclosed herein is also valid in situ.

### Example 8

Physical binding of Aβ globulomer to the P/Q type voltage-gated pre-synaptic calcium channel The Aβ(1-42) globulomers of Reference Example 2 were used as a ligand in an affinity chromatographic approach to identify amyloid-binding proteins isolated from rat brain homogenates. The Aβ(1-42) globulomers were covalently coupled to a suitable matrix, and affinity purified proteins were eluted sequentially and analyzed by mass spec-trometry. This affinity purification resulted in biochemical identification of the Calcium Channel B1, which has 94% identity with the human α1 subunit of the P/Q channel.

a) Immobilization of Aβ(1-42) Globulomers

0.5 ml of commercially available NHS-Sepharose (Pharmacia™, =16-23 μmol/ml) was washed twice with 25 ml of 30% isopropanol in 1 mM HCl and five times with 10 ml of 1 mM HCl. Then the sepharose was washed five times with NHS coupling buffer. All washing steps were performed on ice. Then 650 μl of Aβ(1-42) globulomers of Reference Example 2 and 650 μl of NHS coupling buffer were added to the gel material. Upon incubation at room temperature for 2 h, the suspension was pelleted at 2000 g for 5 min. Free NH₂ groups were blocked with 5 ml NHS blocking buffer for 2 h at room temperature. The Aβ-sepharose was washed with NHS storage buffer and centrifuged. Then 500 μl NHS-storage buffer and 0.02% sodium azide to prevent microbiological growth were added. The suspension was stored at +4° C. until further use.

TABLE 1
Buffers for immobilization.

| NHS-coupling buffer | NHS-blocking buffer | NHS-storage buffer |
|---|---|---|
| 50 mM NaHCO₃ pH 7.5 | 50 mM NaHCO₃ pH 7.5 | 50 mM NaHCO₃ |
| | | pH 7.5 |
| | 250 mM ethanolamine | 0.025% SDS |
| | 0.025% SDS | |

b) Membrane Purification of 50 g Rat Homogenates

Brains were isolated from rats, and 50 g rat brain were added to 450 ml Homogenization Buffer and homogenized with an Ultra Turrax for 20 min at rising speed. The homogenate was centrifuged for 20 min at 2500 rpm (about 1000 g) to remove cell debris. The supernatant was spun down for 25 min at 16000 rpm (about 20000 g) and the pellet was discarded. Next, the 20000 g supernatant was centrifuged for 1 h at 32000 rpm (about 80000 g). The resulting pellets were resupendend with 1 ml PBS each to a final volume of 12.5 ml and pottered with three strokes. The 80000 g supernatant was centrifuged for 1 hour at 43000 rpm (about 150000 g). The 150000 g pellets were resuspended in 500 μl PBS and homogenized by the Ultra Turrax. The 150000 g supernatant was discarded. Subsequently, total protein amount was measured and 11.58 mg/ml protein for the 80000 g fraction and 10.02 mg/ml for the 150000 g fraction were obtained. The proteins of the 80000 g and 150000 g homogenates were solubilized with 2% CHAPS/PBS (20% CHAPS/PBS stock solution) for 16 h at 4° C. The next day the solubilisates were spun at 43000 rpm (about 150000 g) in a TFT 65.13 rotor (Beckman™). The resulting pellet was discarded. The CHAPS solubilisates were resu-pended and diluted 5 fold in PBS to destroy CHAPS micelles. Solubilized proteins were measured to be 0.8 mg/ml for the 80,000 g fraction and 0.57 mg/ml for the 150,000 g fraction. The solutions were stored until further use at −20° C.

TABLE 2
Buffers for membrane homogenates.

| Homogenization Buffer with | |
|---|---|
| protease inhibitor | Protease inhibitors, stock solutions |
| 300 mM Sucrose | 5M diisopropylfluorphosphate (DIFP) |
| 10 mM Tris ph 7.4 | 100 mM N-methylmaleinimid (NEM) |
| 1 mM DIFP | 100 mM EDTA |
| 1 mM EDTA | |
| 1 mM NEM | |
| 10 mg Trypsin inhibitor | |
| from soybeans (Sigma ™) | |

c) Affinity Purification with Immobilized Aβ(1-42) Globulomers

Immobilized Aβ(1-42) globulomers were resuspended and centrifuged at 12,500 rpm for 5 min. The supernatant was discarded and the immobilized globulomers were washed four times with 1 ml PBS. In between, each washing step the suspension was centrifuged for 5 min at 12,500 rpm and the respective supernatant discarded. After that, the globulomers were resuspended in 1×PBS and incubated for 16 h with the CHAPS solubilisates of the 80000 g and 150000 g membrane fraction of rat brain ho-mogenates. Immobilized globulomers were recovered in a Pasteur pipette. Therefore, glass wool was crammed in a Pasteur pipette and rinsed with distilled water. Next, the CHAPS solubilisates containing the immobilized globulomers were poured into the pipette. The immobilisates settled on top of the glasswool while the liquid ran through and was collected in 50 ml Falcon tubes. The Pasteur pipette was washed with 3×0.5 ml PBS/0.4% CHAPS. In order to regain the immobilized globulomers the Pasteur pipette was broken at a height of about 2 cm. The immobilized globulomers were re-suspended in PBS and pipetted quantitatively into an expender tube. PBS was removed by centrifugation at 12,500 rpm for 5 min. Elution and washing steps were performed sequentially as indicated in table 1. After each step, the immobilized globu-lomers were spun down at 12,500 g for 5 min and the supernatant was stored.

TABLE 3
Conditions to elute Aβ(1-42) globulomer-binding proteins.

| Name | Conditions | Volume | | Time | |
|---|---|---|---|---|---|
| Elution 1 | ¼ x PBS 0.05% SDS | 100 | μl | 30 | min |
| Elution 2 | 38/48 kDa globulomers in | 2 × 100 | μl | 2 × 30 | min |
| | ¼ × PBS 0.05% SDS | | | | |
| Wash 1 | 1x PBS | 2 × 225 | μl | 5 | min |
| Elution 3 | PBS 0.5% SDS | 2 × 100 | μl | 30 | min |
| Elution 4 | SDS sample buffer + DTT | 2 × 100 | μl | 30 | min |
| Elution 5 | SDS sample buffer + DTT, | 200 | μl | 5 | min |
| | 95° C. | | | | |

d) SDS-PAGE and Identification of Eluted Proteins by Mass Spectrometry

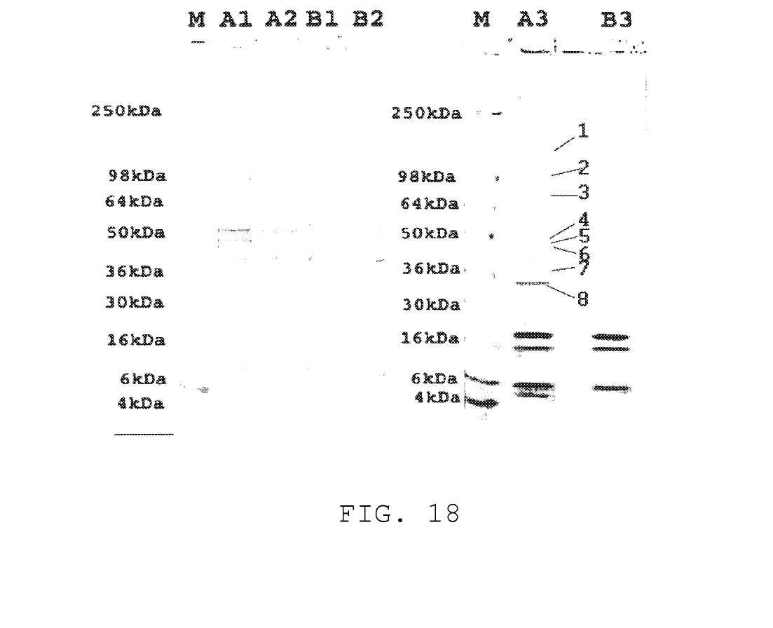

Immobilized Aβ(1-42) globulomers were used as an affinity bait to bind selectively Aβ(1-42) globulomer binding proteins. After distinct washing steps, proteins were eluted with increased stringency. The PBS/0.5% SDS elutions resulted in low protein amounts. In order to obtain significant protein quantities these SDS elutions were concentrated tenfold in centricon tubes. The resulting protein pattern was compared to SDS-patterns and Western Blots of earlier experiments. Special attention was focused on membrane proteins present in the eluates from the 80,000×g fraction. Interesting unknown proteins were selected for further identification by mass spectrometry. FIG. 18 shows the results of the elutions and the selected proteins.

The 4 most abundant bands at 20, 16, 6 and 4.5 kDa were expected to be non-globular Aβ oligomeric forms and were not regarded for analysis. The remaining eight abundant proteins were selected in the 80,000 g fraction for identification by mass spectrometry. After excision, relevant proteins were digested by trypsine. Peptide mass fingerprints were measured by MALDI-TOF-MS. Sequence alignment and database search was performed automatically according to standard procedures (e.g., Martin H Maurer et al. Proteome Science 2003 1, 4). In the following table 4 the identified proteins are listed.

TABLE 4
Amyloid-binding proteins determined by 38/48
kDa Aβ₁₋₄₂ globulomers affinity chromatography

| | Observed | Theroretical. | | |
|---|---|---|---|---|
| | Molecular | Molecular | NIH accession | |
| Band | Weight (Da) | Weight (Da) | number | Protein Name |
| 1 | 165500 | 188658 | gi201138801 | Intersectin 2 (SH3 domain- |
| | | | | containing protein 1B) (EH and |
| | | | | SH3 domains protein 2) EH |
| | | | | domain and SH3 [Mus musculus] |
| | | 167378 | gi16924000 | densin-180 [Rattus norvegicus] |
| | | 175804 | gi16758820 | RIM2 protein [Rattus |
| | | | | norvegicus] |
| 2 | 95000 | 94325 | gi1352648 | Osmotic stress protein 94 (Heat |
| | | | | shock 70-related protein APG-1) |
| | | | | [Mus musculus] |
| | | 122522 | gi17656906 | activin receptor interacting pro- |
| | | | | tein |
| 3 | 83000 | 84891 | gi13878219 | ribonuclease/agiogenin inhibitor |
| | | | | 2 [Mus musculus] |
| 4 | 49000 | 51506 | gi6651165 | syndapin IIab [Rattus |
| | | | | norvegicus] |
| | | 48496 | gi17477318 | similar to K-depended Na/Ca |
| | | | | exchanger NCKX4 [Homo |
| | | | | sapiens] |
| | | 4511 | gi423742 | beta-amyloid [guinea pig] |
| | | | | (fragment 1-42) |
| 5 | 48000 | 41724 | gi71620 | actin beta [Rattus norvegicus] |
| | | 257186 | Gi2136947 | calcium channel BI1 [Rabbit] |
| | | 4511 | gi423742 | beta-amyloid protein [guinea |
| | | | | pig] (fragment 1-42) |
| 6 | | | | Not determined |
| 7 | 38000 | 4511 | gi423742 | beta-amyloid protein [guinea |
| | | | | pig] (fragment 1-42) |
| 8 | | | | Not determined |

Ten different proteins were detected in the analyzed positions. Band 6 and band 8 could not be matched to a protein in the database. In band 5 the calcium Channel BI1 was detected at an apparent molecular mass in SDS-PAGE of 48 kDa. Given the theoretical molecular weight of 257 kDa and the NIH accession number gi2136947 (equals P27884 in Swiss Prot data base) this is a fragment of the Voltage-dependent P/Q-type calcium channel subunit alpha-1A. Synonyms are:
Voltage-gated calcium channel subunit alpha Cav2.1
Calcium channel, L type, alpha-1 polypeptide isoform 4
Brain calcium channel I
BI

Due to high sequence homology between rabbit and rat species the computer search lead to the rabbit protein although the experiment was performed with rat brain ho-mogenate. Thus, it was demonstrated that Aβ(1-42) globulomer is capable of physically binding to the P/Q type voltage-gated presynaptic calcium channel.

### Second Series of Experiments

### Reference Example 9

### Cell Culture

Primary hippocampal cell cultures were prepared from Wistar rat embryos at the embryonic age E19 in accordance with the protocol described earlier by Banker and Cowan (1977). Briefly, pregnant rats were deeply anesthetized by ether narcosis and decapitated. Embryos were rapidly removed and brains were dissected under constant cooling with chilled (˜4° C.) phosphate buffered saline (PBS). Then both hippocampi were taken out and washed twice with ice-cold PBS followed by a wash with PBS at room temperature. Hippocampi were triturated using three siliconized pipettes with decreasing tip diameters. Cells were then plated on coverslips (density 60000 cells/coverslip) coated with 0.01% poly-L-lysine solution and stored at 37° C. in an incubator gassed with 5% CO₂ in normal air. The culture medium contained 0.25% penicil-line/streptomycine, 2% B27, 0.25% L-glutamine (Gibco, Karlsruhe, Germany).

Throughout culturing, we added 0.5 μM/L ω-conotoxin MVIIA to the culture medium to block N-type calcium channels and to stabilize the expression of P/Q-type currents. Cells were cultured for 14 to 28 days until used for experiments.

### Reference Example 10

### Current Recording

Currents were measured under whole-cell voltage-clamp conditions at room temperature using borosilicate pipettes of 2-4 MΩ resistance. Electrode solution contained (in mM/l): NaCl 10, KCl 100, CaCl₂ 0.25, EGTA 5, HEPES 10, glucose 40 (pH set at 7.3) when used for recordings of synaptic events. A low-chloride solution was used for experiments in which GABA induced currents were elicited, which consists of (mM): Cs-gluconate 130, CsCl 10, CaCl₂ 0.5, MgCl₂ 2, EGTA 10, HEPES 10, Mg-ATP 2 (pH: 7.3). Using this solution the calculated equilibrium potential for chloride-ions was −54 mV. During calcium current measurements the solution contained in (mM): CsCl 110, EGTA 10, HEPES 25, tris-phosphocreatine 10, Mg-ATP 4, Na-GTP 0.3 and 20 units/ml creatine-phosphokinase at pH 7.3. Osmolarity was adjusted to 295 mosmol/l, when necessary, by adding glucose. Bath solutions contained (in mM): NaCl 156, KCl 2, CaCl₂ 2, MgCl₂, Glucose 16.5, HEPES 10 (pH set to 7.3) for recordings of synaptic events and TEA-CI 140, BaCl₂, 10, HEPES 10, and Glucose 20 at a pH: 7.3 for calcium currents, respectively. The bath perfusion was stopped for 10 min prior to the application of the Aβ(1-42) globulomer and baseline activity was recorded. Subsequently, Aβ(1-42) globulomer (164 nM in respect to the 12mer complex) was added to the bath by means of a micro pump, yielding a final concentration of 82 nM. TTX, ω-agatoxin IVA, ω-conotoxin MVIIA, roscovitine (Alomone Labs, Jerusalem, Israel), and nifedipine (Sigma, Deisenhofen, Germany) were added directly to the bath solution at the concentrations indicated.

Currents were measured with an Axopatch 200B (Axon Instruments, Union City, US) or an EPC-7 amplifier (HEKA, Lambrecht, Germany), digitized by a CED 1401 micro analog/digital converter (CED, Cambridge, UK) and stored on a PC (sample frequency 20 kHz). All recorded currents were low-pass filtered with a cut-off frequency of 3 kHz. Capacitive transients and series resistances were compensated on-line (˜50-60% compensation) during the calcium current measurements. No compensation was performed during recordings of synaptic events. Data were evaluated off-line using Spike5 and Signal3 software (CED, Cambridge, UK). All calcium current traces were corrected for aspecific linear leak (reversal potential 0 mV) determined at holding potential using ±5 mV potential steps.

### Reference Example 11

### Current Analysis

All cells were voltage clamped at a holding potential of −80 mV, and calcium ions were substituted by Barium ions to increase the amplitude of the current flow through the calcium channels. Peak amplitudes of the currents (I) evoked with the activation protocol were plotted as a function of membrane potential (V). The resulting IV-relations were fitted with a combination of a first order Boltzmann activation function and the Goldman-Hodgkin-Katz (GHK) current-voltage relation (Kortekaas and Wadman, 1997):

$$I ( V ) = V \frac{{g}_{max}}{1 + exp ( \frac{{V}_{h} - V}{{V}_{c}} )} \frac{{[ {Ba}^{+} ]}_{in} / {[ {Ba}^{+} ]}_{out} - exp ( - α V )}{1 - exp ( - α V )} \qquad [ 1 ]$$

with α=F/RT and gₘₐₓ=α FP₀ [Ba⁺]ₒᵤₜ,

where gₘₐₓ is the maximal membrane conductance (which is proportional to the maximal permeability and the extracellular concentration of barium), Vₕ is the potential of half maximal activation and V_{c} is proportional to the slope of the curve at Vₕ. F represents the Faraday constant, R the gas constant, P₀ is the maximal permeability, and T the absolute temperature. The intracellular concentration of Ba²⁺ was assumed to be 0.01 μM. Assuming higher values of up to 0.1 mM did not significantly change the resulting values of the parameters.

The voltage dependence of steady state inactivation of the barium current was estimated from the relation of peak current amplitude versus the pre-potential. This relation was well described by a Boltzmann function, which normalized the current:

$$N ( V ) = \frac{I ( V )}{{I}_{max}} \\ where \\ I ( V ) = \frac{{I}_{max}}{1 + exp ( \frac{{V}_{h} - V}{{V}_{c}} )} \qquad [ 2 ]$$

where N(V) is the level of steady state inactivation determined from the current amplitude I(V) normalized to Iₘₐₓ, V is the pre-pulse potential, Vₕ is the potential of half maximal inactivation and V_{c} is a factor proportional to the slope of the curve at Vₕ.

### Reference Example 12

### Synaptic Events

For these recordings, all cells were voltage clamped at a holding potential of −70 mV. Synaptic events triggered by the release of GABA were inwardly directed (E_{α}˜−10 mV) due to the use of high chloride concentrations in the pipette and the bath. Routinely, 10 min of baseline activity was acquired, serving as control data, before any drug application was started. Synaptic events were then analyzed off-line for frequency and amplitude, using a custom-made, template based algorithm.

### Reference Example 13

### Statistics

Values are presented as the mean±standard error of the mean (SEM). Statistical comparisons were made with Student's t-test. A p-value <0.05 was used to indicate significant differences.

### Example 14

### Aβ(1-42) Globulomer Reduces Spontaneous Synaptic Activity in Hippo-Campal Cell Cultures

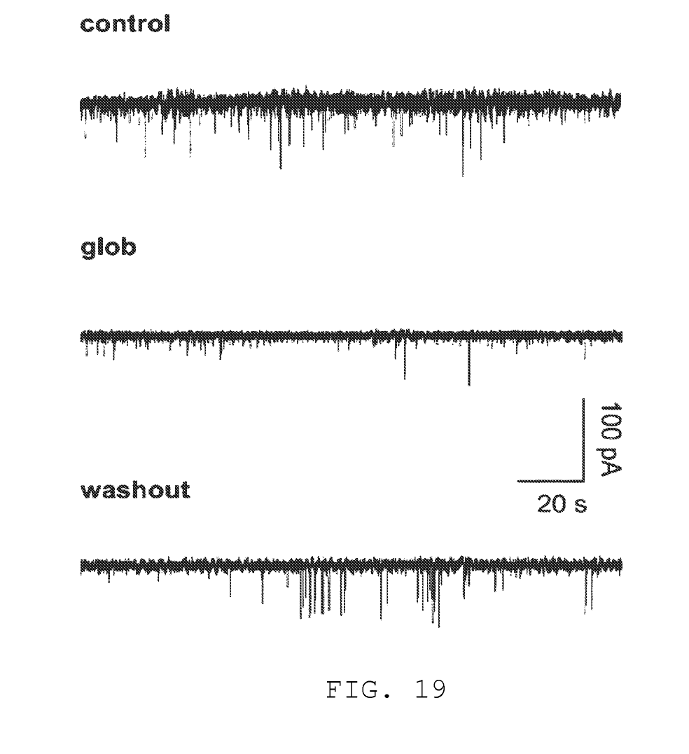

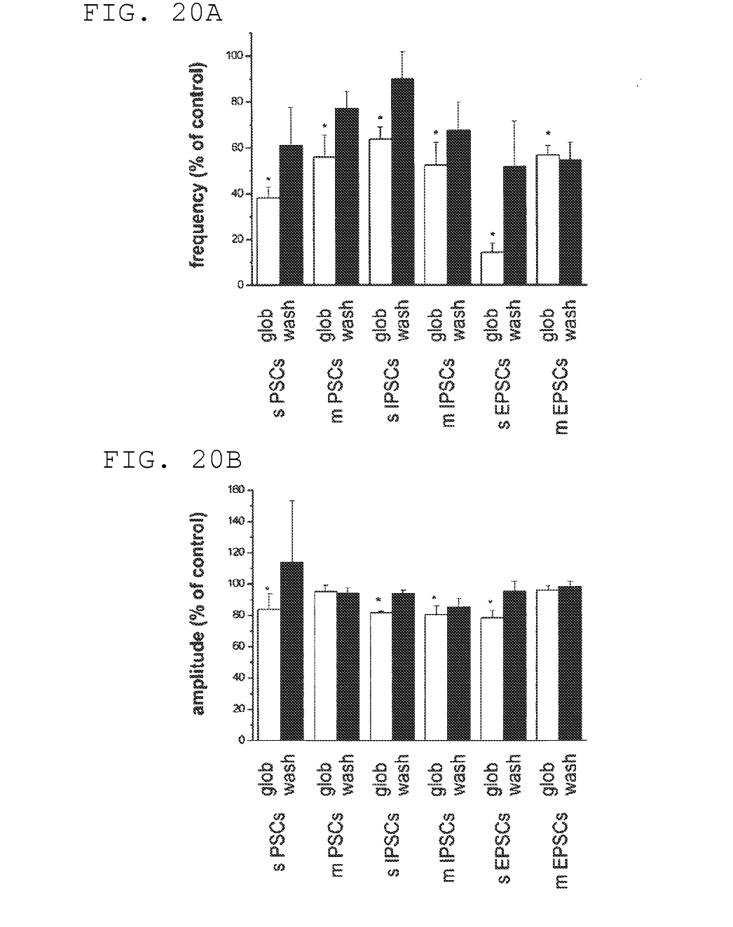

Spontaneous synaptic was measured activity in cultured hippocampal neurons using whole-cell voltage clamp techniques (V_{hold}=−70 mV). Under our ionic conditions, all synaptic events appeared as inward currents (spontaneous postsynaptic currents; sPSCs) with a mean frequency of 189±63/min (n=13). Bath-application of 82 nM Aβ(1-42) globulomer (globulomer molarities calculated with respect to the 12mer complex) rapidly reduced the frequency of sPSCs to 38±5% of control (p<0.05; n=13; FIG. 19). This effect was partially reversible upon washout in 2 of 3 cells tested (61±16%). The median amplitude of events was 310±168 pA and was reduced to 84±10% under Aβ(1-42) globulomer (p<0.05; n=14; FIG. 20). Similar—but slightly weaker—effects were seen after application of 8.2 nM Aβ(1-42) globulomer (frequency reduced to 63±9%; p<0.05; median amplitude 94±5% of control, n=8, n.s.). Thus, the suppression of spontaneous synaptic activity by Aβ(1-42) globulomer is dose-dependent and starts at low nanomolar concentrations. Input resistance was not affected by Aβ(1-42) globulomer (control: 120.9±13.6 MΩ; Aβ(1-42): 131.6±13.7 MΩ).

Suppression of synaptic currents by an agent may be caused by changes in neuronal activity or, alternatively, by specific synaptic interactions. It was therefore tested for effects of Aβ(1-42) globulomer on active discharge properties by recording action potentials in current clamp mode. Action potentials elicited by current injection showed no difference in amplitude, shape or kinetics when compared before and after Aβ(1-42) globulomer application. In detail, the threshold for firing was −22.5±8.2 mV vs. −24.2±9.8 mV, and the amplitude of the Aβ (baseline to peak) amounted to 119.9±11.2 vs. 1 10.9±16.7 mV. Likewise, kinetic parameters did not differ: values for the half-width time were 3.5±1.6 ms vs. 4.0±2.9 ms, maximal rate of rise 100.5±46.4 V/s vs. 84.2±50.0 V/s and maximal rate of repolarization 46.0±18.6 V/s vs. 47.4±19.3 V/s (n=16 action potentials from 4 cells before and after Aβ(1-42) globulomer respectively. It thus appears that the alteration of synaptic activity by Aβ(1-42) globulomer may be caused by a direct interaction with pre- or postsynaptic proteins, rather than by an un-specific alteration of cellular excitability.

This hypothesis was corroborated by recordings of spontaneously occurring miniature postsynaptic currents (mPSCs) in the presence of TTX. Similar to spontaneous “macroscopic” PSCs, these currents were reduced in frequency by 82 nM Aβ(1-42) globu-lomer (yielding 56±9% of control; p<0.05; FIG. 20). However, the amplitude of mPSCs was unaltered (median amplitude 31.1±4.0 pA under control conditions vs. 30.2±5.2 pA in the presence of Aβ(1-42) globulomer, n=6). Upon washout for 10 minutes, the effect on event frequency recovered partially to 77±7.6% of control, n=6, wash: 4/6). Together, these data suggest that Aβ(1-42) globulomer interferes with the presynaptic machinery of transmitter release.

### Example 15

### Effects on Spontaneous and Miniature Inhibitory Postsynaptic Currents

Pharmacologically naive synaptic currents reflect a mixture of glutamatergic (excitatory) and GABAergic (inhibitory) events. In order to differentiate between these components, inhibitory postsynaptic currents were isolated by adding CNQX (20 μM) and DL-APV (30 μM) to the bath solution. The frequency of spontaneously occurring IPSCs was suppressed by Aβ(1-42) globulomer (yielding 64±5% of control; p<0.05; n=12) and the median amplitude was reduced to 82±2% of control (p<0.05). These reductions could be reversed to some degree following withdrawal of the globulomer (frequency: 90±12%; amplitude: 94±2%). Miniature inhibitory postsynaptic currents (mlPSCs, recorded in 0.5 μM TTX) did also show a similar reduction of frequency after application of Aβ(1-42) globulomer (52±10% of control; p<0.05; n=6). This effect was partially reversible upon washout, yielding 68±12% of control (FIG. 20). In addition, a reduction of mlPSC amplitude was observed (81±6% of control; p<0.05; no washout in 3/3 cells (85±6%)).

### Example 16

### Effects on Postsynaptic GABA_{A} Receptors

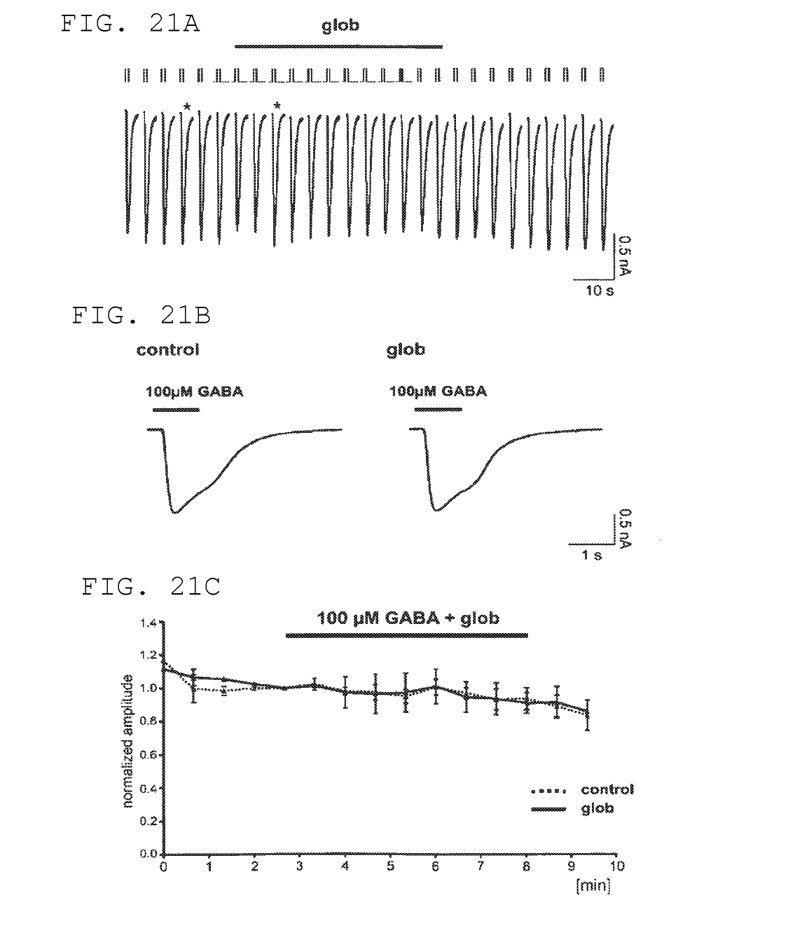

In order to test for potential effects of Aβ(1-42) globulomer on postsynaptic GABA_{A} receptors, a high (100 μM) concentration of GABA was applied by brief pressure-pulses directly onto the cell. Repetitive application of GABA to cultured cells elicited large (>1 nA) inward currents which showed only minor rundown with time. This behaviour was unaltered after application of Aβ(1-42) globulomer for 5 min, indicating that GABA_{A} receptors are not affected by the agent (FIG. 21).

### Example 17

### Effects on Spontaneous and Miniature Excitatory Postsynaptic Currents

Finally, excitatory synaptic currents (EPSCs) were isolated in the presence of 5 μM gabazine (a GABA_{A} receptor antagonist). Basal frequency of these events was 386±124/min. Their frequency was reduced by Aβ(1-42) globulomer to 14±4% of control (p<0.05; n=6; FIG. 20). Likewise, the amplitude was reduced to 79±4% of control (n=6; p<0.05; FIG. 20). The effect was partially reversible during washout (frequency increasing to 52±19% of control, amplitude to 96±6%; n=6). The frequency of miniature EPSCs was likewise suppressed to 57±9% of control (n=6; p<0.05), while the amplitude of mEPSCs remained stable (96±3% of control). The frequency suppression did not recover upon wash-out (54±8%; n=6).

Together, these data indicate that Aβ(1-42) globulomer depresses vesicular release at GABAergic and glutamatergic synapses, most likely by decreasing the probability of vesicle exocytosis from presynaptic terminals.

### Example 18

### Effects on Voltage-Activated Calcium Currents

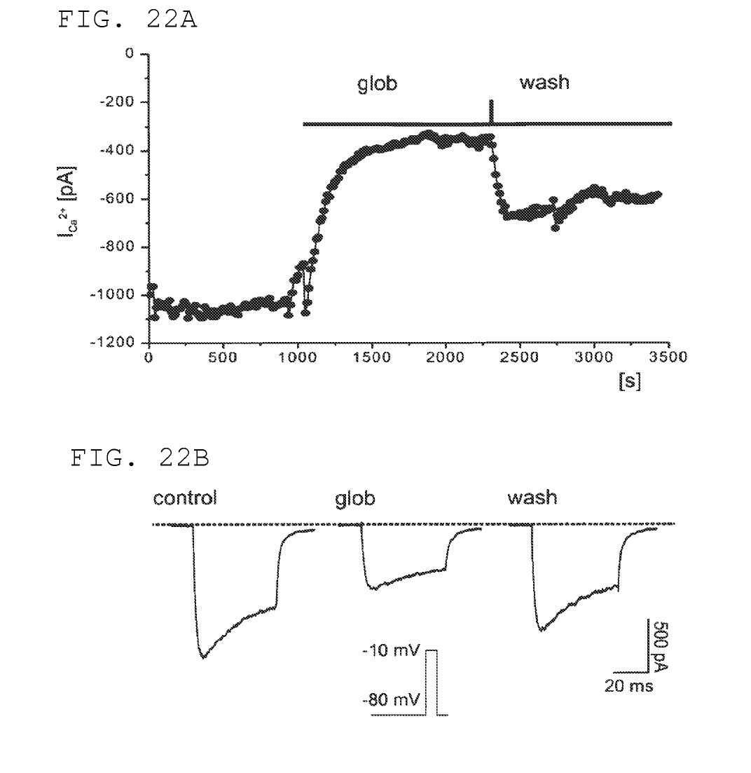

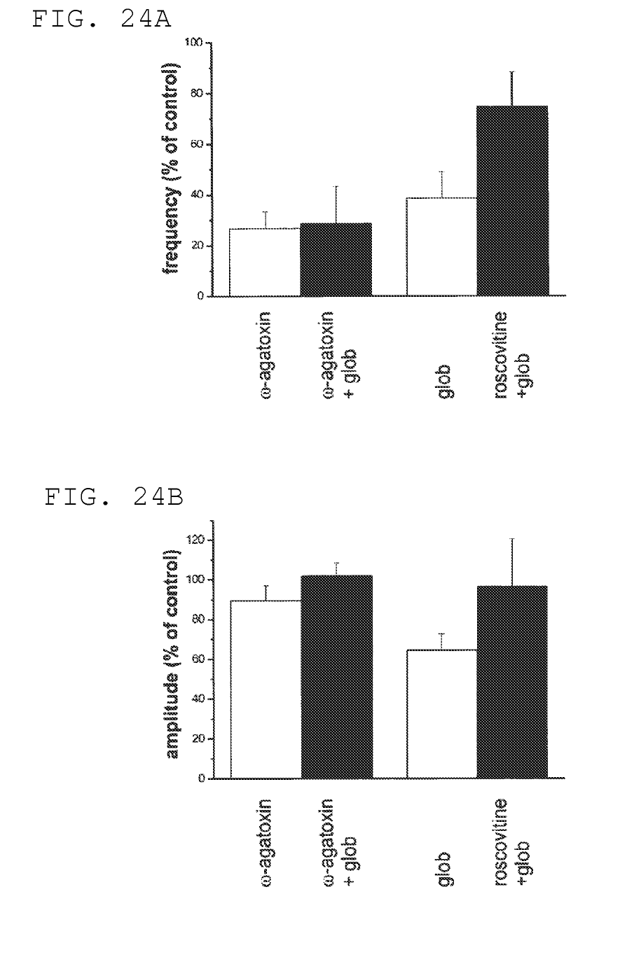

Presynaptic vesicle release is triggered by an influx of calcium into the presynaptic terminal. Therefore, Aβ(1-42) globulomer might act on presynaptic calcium signalling. A common pathway for release of both, glutamatergic and GABAergic vesicles is presynaptic calcium influx via N-type or P/Q-type calcium channels. Therefore, the effects of Aβ(1-42) on whole-cell calcium currents in cultured hippocampal neurons were ana-lyzed. Typical P/Q channel-mediated currents could be reliably elicited in somatic whole-cell recordings under our culture conditions. In these experiments, 10 mM Ba²⁺ was used as charge carrier in the extracellular solution (see methods). Measurements were performed in the presence of 10 μM nifedipine (a L-type calcium channel blocker), co-conotoxin MVIIA (a N-type calcium channel blocker) and blockers of other voltage-gated ion channels (TTX 0.5 μM, TEA 140 mM, Cs⁺-based intracellular solution). Rundown of these currents was avoided by adding 20 U/ml phosphocreatine kinase and 10 mM tris-phosphocreatine to the pipette solution. Under these conditions, P/Q-type currents were evoked by a depolarizing voltage step to −10 mV (mean amplitude 1015±145 pA; FIG. 22). Aβ(1-42) globulomer reduced the amplitude of these currents to 62±7% of control (n=10). This effect was partially reversible in 3/3 cells. If the effect of Aβ(1-42) globulomer on synaptic currents is mediated by block of P/Q-type calcium channels, it should be mimicked and occluded by the selective P/Q-type calcium channel blocker ω-Agatoxin IVA. Indeed, this toxin (0.5 μM) reduced the frequency of miniature PSCs to 27±7% (n=3; amplitude 90±7%), similar to the effect of Aβ(1-42) globulomer. Following pre-incubation with ω-Agatoxin IVA, Aβ(1-42) globulomer had no additional effect on the synaptic currents (n=6, frequency 108±15%; amplitude 102±7% of currents after ω-Agatoxin IVA control; FIG. 24). These data suggest that ω-Agatoxin IVA and Aβ(1-42) globulomer share the same molecular mechanism.

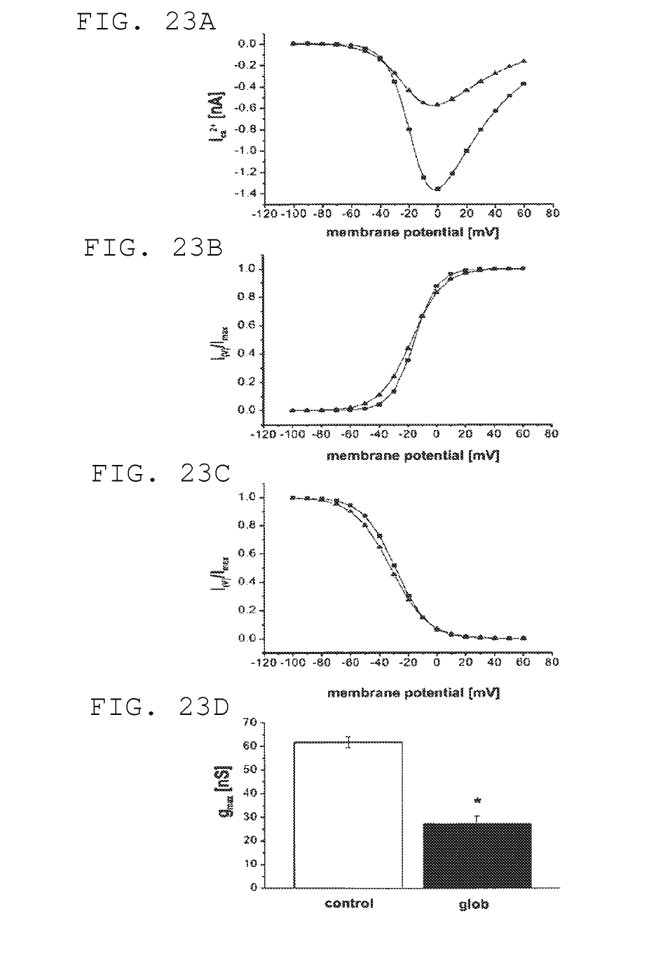

The effect of Aβ(1-42) globulomer on P/Q-type calcium currents was further character-ized by steady-state activation and -inactivation protocols (see methods). Maximal conductance (gₘₐₓ) was 61.7±2.4 nS (control) versus 27.2±3.2 nS (Aβ(1-42) globulomer, p<0.05; n=6; FIG. 23). Thus, Aβ(1-42) globulomer reduces the current amplitude without affecting its voltage-dependent activation. In contrast to this marked reduction in conductance (and current amplitude), other kinetic parameters were not affected by Aβ(1-42) globulomer. Steady-state activation was characterized by Vₕ=−15.4±1.1 mV which was not changed after application of Aβ(1-42) globulomer (Vₕ=−17.3±1.3 mV; n=6). The slope of the fitted first-order Boltzmann-equation V_{c} was −7.8±0.3 mV in control solution and −10.8±0.5 mV in Aβ(1-42) globulomer (not different, n=6). Likewise, steady-state inactivation was not affected by Aβ(1-42) globu-lomer, as indicated by stable values for the voltage at half-maximal inactivation (29.2±0.6 mV in control; 32.4±1.2 mV in Aβ(1-42) globulomer; n=4) and for the slope V₀ (−11.0±0.9 mV versus −12.6±1.1 mV; FIG. 23).). Thus, Aβ(1-42) globulomer reduces the current amplitude without affecting its voltage-dependent activation or inactivation.

In addition the effects of Aβ(1-42) globulomer on N- and L-type calcium currents were analyzed. Besides blockers for Na⁺- and K⁺-channels (see above) 0.5 μM ω-agatoxin IVA were added to block P/Q-channels. L-type calcium currents were isolated by addition of 0.5 μM ω-conotoxin MVIIA. Voltage pulses from −80 mV to −10 mV elicited inward currents of 597.7±230.9 pA amplitude which remained stable after addition of Aβ(1-42) globulomer (573.0±225.6 pA; n=3). When N-type currents were isolated by adding nifedipine (10 μM) instead of ω-conotoxin, the same voltage clamp protocol elicited inward currents which were, again, insensitive to Aβ(1-42) globulomer (amplitude in control solution 1368.9±332.8; amplitude in Aβ(1-42) globulomer 1399.8±376.4 pA; n=3). When all blockers were added together, the remaining calcium current (possibly R-type) was too small for a detailed analysis (<100 pA), indicating that this component was only marginally expressed in the cultured hippocampal neurons.

### Example 19

### Rescue by Roscovitine

Application of roscovitine in the presence of Aβ(1-42) globulomer did indeed partially recover the frequency of synaptic currents. While in these experiments Aβ(1-42) globulomer reduced the frequency of spontaneous PSCs to 38±10% of control, application of roscovitine (20 μM) brought this parameter back to 75±13% (n=5; FIG. 24).

Together, these data indicate that Aβ(1-42) globulomer reduces the frequency of spontaneous and miniature synaptic currents by suppression of presynaptic calcium influx via P/Q-type calcium channels.

### Third Series of Experiments

### Example 20

### Enhancing P/Q Calcium Currents by Roscovitine Prevents/Reverses Chronic Aβ Globulomer-Induced Deficits on Evoked Synaptic Tranmission in Hippocampal Tissue

Rat hippocampal slice cultures (9 days old Wistar rats; 15-17 DIV) were incubated over night with either Aβ(1-42) globulomer (at a concentration corresponding to approximately 1 μM of Aβ monomer), Aβ(1-42) globulomer (at a concentration corresponding to approximately 1 μM of Aβ monomer)+20 μM roscovitine, or control (SDS). Recordings were performed (in artificial cerebrospinal fluid) from CA1 stratum radiatum after stimulation of the Schaffer collateral at different intensities.

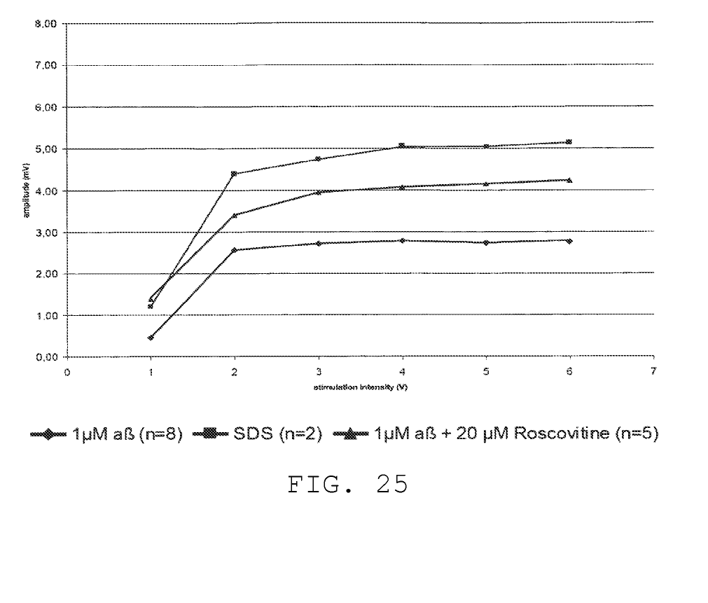

Results are shown in FIG. 25, demonstrating that the application of globulomer strongly suppresses synaptic transmission. Co-application of 20 μM roscivitine completely prevents/reverses the globulomer-induced deficit.

### Example 21

### Effect of Extracellular Ca²⁺ on sPSC Frequency after Treatment with Aβ(1-42) Globulomer

Spontaneous synaptic activity was measured in cultured hippocampal neurons using whole-cell voltage clamp techniques (V_{hold}=−70 mV). Under the ionic conditions used (E_{C1}˜−10 mV) all synaptic events appeared as inward currents.

The effects of Aβ(1-42) globulomer (at a concentration corresponding to approximately 1 μM of Aβ monomer) were assessed by comparing spontaneously occurring postsynaptic currents (sPSCs) in single cells in 5 min intervals in the presence or absence of globulomer in bath solution containing 1 mM Ca²⁺. Currents recorded prior to the addition of the globulomer served as control describing basal synaptic transmission. Currents recorded in the interval immediately after application were analysed with respect to the control data. Afterwards, extracellular Ca²⁺ was elevated from 1 mM to 4 mM (leaving the concentration of globulomer unchanged). Currents in the following 5 min recording interval were again analysed with respect to control data.

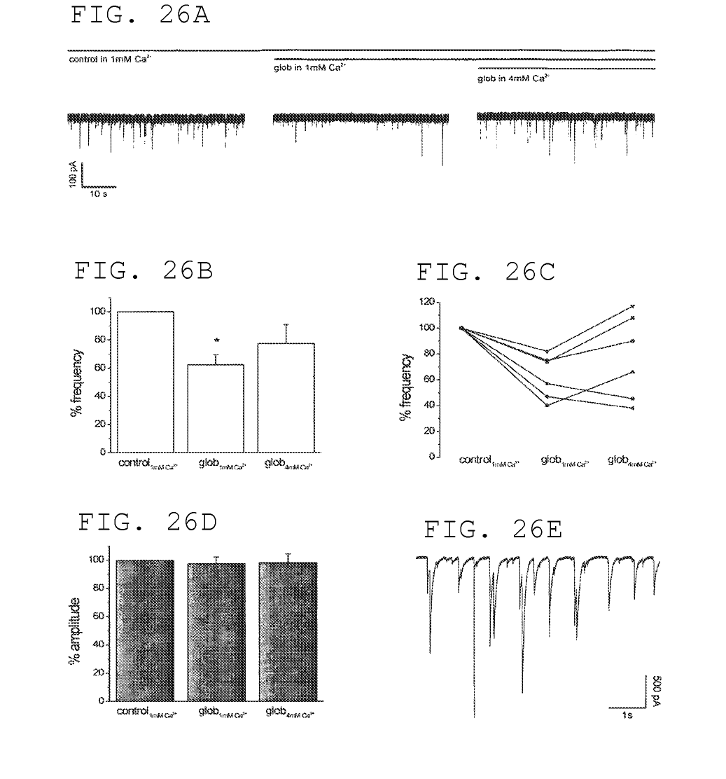

Basal frequency of sPSCs in 1 mM Ca²⁺ was 4.2±1.2 Hz. Bath-application of Aβ(1-42) globulomer rapidly reduced the sPSC frequency to 63±7% of control (p<0.05; n=6; FIG. 26 A+B). After elevation of extracellular Ca²⁺ to 4 mM, sPSC frequency partially recovered to 77±13% of control (FIG. 26 B). In 4 of 6 cells tested, sPSC frequency increased, whereas it remained unaltered in the other 2 cells (FIG. 26 C).

Median amplitude of sPSCs under control conditions was 27.7±2.2 pA and remained unaltered after addition of Aβ(1-42) globulomer (97±5%; FIG. 26 D) or subsequent elevation of extracellular Ca²⁺ (98±6%).

In most cases, prominent currents with amplitudes up to 2000 pA occurred directly after elevation of extracellular Ca²⁺-concentration. These currents with multiple peaks (see FIG. 26 E) were rejected from analysis.

This clearly demonstrates that the principle of activating the P/Q type presynaptic calcium channel is effective in compensating the detrimental effects exerted by Aβ globulomer.

### Example 22

### Blocking P/Q Voltage-Gated Presynaptic Calcium Channels with Anti-P/Q Type Antibody Prevents Chronic Aβ Globulomer-Induced Deficits on Evoked Synaptic Tranmission in Hippocampal Tissue

Rat hippocampal slice cultures (9 day old Wistar rats; 15-17 DIV) were incubated over night with either Aβ(1-42) globulomer (at a concentration corresponding to approximately 1 μM of Aβ monomer), Aβ(1-42) globulomer (at a concentration corresponding to approximately 1 μM of Aβ monomer)+polyclonal antibody serum against the P/Q type voltage-gated presynaptic calcium channel antibody (sc-16228; 0.3 μg/ml=approximately 2 nM), or control (SDS). The antibody is an affinity purified goat polyclonal antibody raised against a peptide mapping near the C-terminus of the α1A subunit of the P/Q type voltage-gated presynaptic calcium channel of human origin. It is commercially available from Santa Cruz Biotechnology, Inc. Recordings were performed (in artificial cerebrospinal fluid) from CA1 stratum radiatum after stimulation of the Schaffer collateral at different intensities.

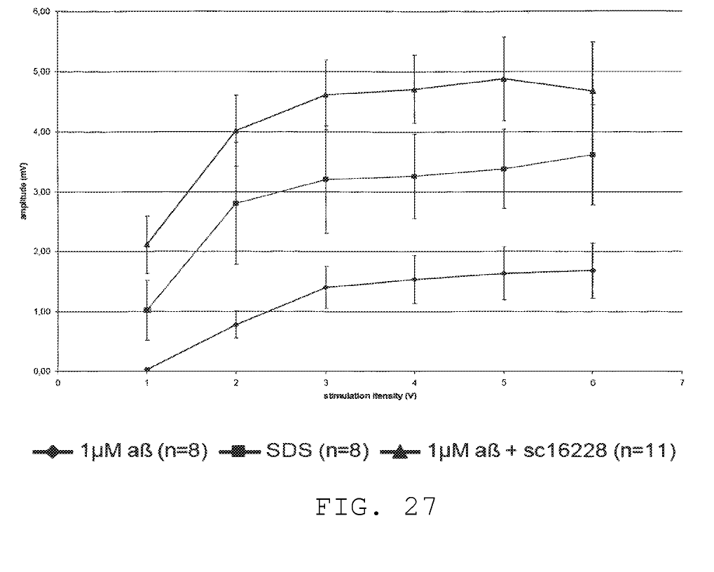

Results are shown in FIG. 27, demonstrating that the application of globulomer strongly suppresses synaptic transmission. Co-application of the antibody completely prevents the globulomer-induced deficit.

### Example 23

### Lack of Effect of Monomeric Aβ(1-42) Peptide on mPSCs

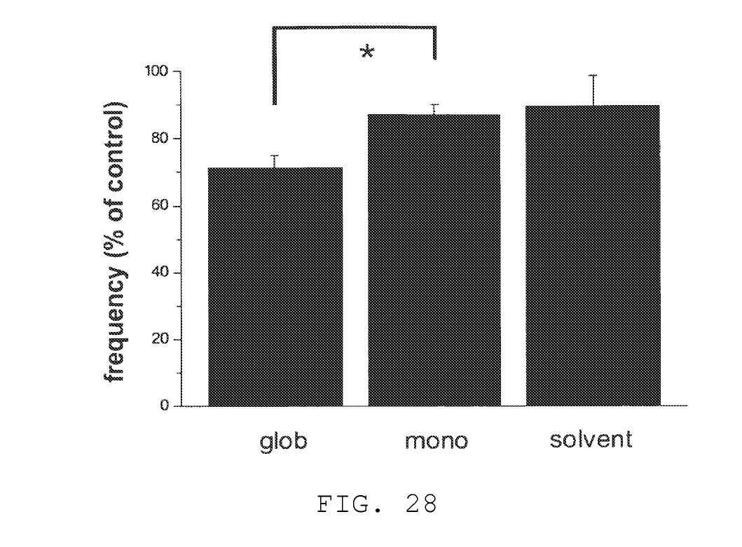

To test for the specificity of the Aβ(1-42) globulomer effect, a preparation of synthetic monomeric Aβ(1-42) peptide was applied while recording mPSCs in the presence of TTX. A temporarily stable monomer solution was prepared by dissolving synthetic Aβ(1-42) in 0.1% NaOH (see reference example 2). A Coomassie-stained SDS-PAGE confirmed the presence of Aβ(1-42) monomer and the Aβ(1-42) globulomer at the expected molecular weights in the respective preparations. The monomeric preparation was bath-applied at an initial concentration of 1 μM Aβ(1-42) monomer, which equals the amount of monomer contained in the globulomer preparation. Frequency of mPSCs was 87±3% of control in the presence of monomeric Aβ(1-42) (n=7; n.s.) (FIG. 28), which was similar to the frequency observed by application of the solvent alone (0.1% NaOH diluted 1:1000 in bath solution; 89±9% of control; n=7) (FIG. 28). The amplitude of mPSCs was unaltered after application of the monomer preparation (median amplitude, 34.2±3.0 pA under control conditions vs 33.7±3.0 pA in the presence of Aβ(1-42) monomer) or its respective solvent (median amplitude, 32.4±1.5 pA under control conditions vs 32.3±1.1 pA in the presence of the solvent).

Note that, in general, Aβ(1-42) peptide can hardly be maintained in its monomeric state in physiological buffers, because it aggregates within minutes to protofibrils and fibrils. 0.1% NaOH was used as the initial solubilization buffer for the synthetic Aβ(1-42) pep-tide, which is the most suitable buffer for solubilising and maintaining Aβ(1-42) peptide in a monomeric state under the experimental conditions. Although great care was taken to minimize Aβ(1-42) peptide aggregation, aggregation was observed at the final dilution of 0.0001% NaOH in the bath solution when samples were retrieved after the actual experiments. Therefore, the applied monomeric Aβ(1-42) peptide is likely a mixture of Aβ(1-42) aggregation states (i.e., Aβ(1-42) monomer, Aβ(1-42) protofibrils, and Aβ(1-42) fibrils). Furthermore, aggregated Aβ(1-42) peptide within the monomeric Aβ(1-42) preparation can also be seen in the SDS-PAGE gel loading pocket. Preparations of Aβ(1-42) tend to adhere to surfaces and therefore may reach lower final effective concentrations at the target cells. Therefore, the Aβ(1-42) content was representa-tively determined after the experiment and it was found that in both Aβ(1-42) monomer and globulomer preparations, >50% of the initial Aβ(1-42) peptide were present during the electrophysiological recordings.

1-14. (canceled)

15. A method for the treatment of an amyloidosis disease comprising administering to a subject in need thereof an agent that inhibits the interaction between Aβ globulomer and P/Q type voltage-gated presynaptic calcium channel, wherein the agent is an antibody that binds to the Aβ globulomer or the P/Q type voltage-gated presynaptic calcium channel.

16. The method of claim 15, wherein the amyloidosis disease is selected from the group consisting of Alzheimer's disease and Down's syndrome.

17. The method of claim 15, wherein the inhibitor reduces the interaction between Aβ globulomer and the P/Q to less than one half of its normal value.

18. The method of claim 15, wherein the treatment is for the restoration of synaptic function.

19. The method of claim 15, wherein the treatment is for the restoration of long-term potentiation.

20. The method of claim 15, wherein the treatment is for the restoration of memory function.

21. The method of claim 15, wherein the treatment is for the restoration of performance of activities of daily living (ADL) capacity.

22. A method for identifying an inhibitor of the interaction between Aβ globulomer and the P/Q type voltage-gated presynaptic calcium channel, comprising determining the inhibitory effect on the interaction between Aβ globulomer and the P/Q type voltage-gated presynaptic calcium channel of said agent.

23. The method of claim 22, comprising determining the effect of said agent on a cell expressing at least the P/Q type voltage-gated presynaptic calcium channel and an intracellular calcium sensor.

24. The method of claim 22, comprising determining the physical contact between Aβ globulomer and the P/Q type voltage-gated presynaptic calcium channel.
